(19) United States
(12) Patent Application Publication
(10) Pub. No.: US 2009/0304666 A1
(43) Pub. Date: Dec. 10, 2009

(54) CONJUGATE FOR THE SPECIFIC TARGETING OF ANTICANCER AGENTS TO TUMOR CELLS OR TUMOR VASCULATURE AND PRODUCTION THEREOF

(75) Inventors: Roger G. Harrison, Norman, OK (US); J. Thomas Pento, Norman, OK (US); Stuart E. Lind, Greenwood Village, CO (US)

Correspondence Address: DUNLAP CODDING, P.C., PO BOX 16370, OKLAHOMA CITY, OK, 73113, US

(73) Assignee: The Board of Regents of the University of Oklahoma

(21) Appl. No.: 12/502,011

(22) Filed: Jul. 13, 2009

Related U.S. Application Data
(63) Continuation of application No. 11/712,140, filed on Feb. 28, 2007.
Application No. 11/712,140 is a continuation-in-part of application No. 10/870,832, filed on Jun. 17, 2004, now abandoned.
(60) Provisional application No. 60/777,725, filed on Feb. 28, 2006.
(60) Provisional application No. 60/479,106, filed on Jun. 17, 2003.

(51) Int. Cl. A61K 38/51; A61P 35/00; C12N 5/06

(52) U.S. Cl. 424/94.5; 435/375

(57) ABSTRACT

A conjugate is disclosed herein, wherein the conjugate includes a ligand having the ability to specifically and stably bind to an external receptor or binding site on a tumor vasculature endothelial cell, wherein the external receptor or binding site is specific for tumor vasculature endothelial cells. The conjugate also includes an anticancer agent that is selectively toxic to cancer cells operatively attached to the ligand. The anticancer agent may be L-methioninase. Pharmaceutical compositions comprising the conjugate are also disclosed, as well as methods of treating a cancer tumor or cancer cells with a therapeutically effective amount of the conjugate.

### CROSS REFERENCE TO RELATED APPLICATIONS

This application is a continuation of U.S. Ser. No. 11/712,140, filed Feb. 28, 2007; which claims benefit under 35 U.S.C. 119(e) of U.S. Provisional Application No. 60/777,725, filed Feb. 28, 2006. This application is also a CIP of U.S. Ser. No. 10/870,832, filed Jun. 17, 2004; which claims benefit of U.S. Provisional Application No. 60/479,106, filed Jun. 17, 2003. The contents of each of the above-referenced patent applications are hereby expressly incorporated in their entirety herein by reference.

### STATEMENT REGARDING FEDERALLY SPONSORED RESEARCH OR DEVELOPMENT

Not applicable.

### BACKGROUND OF THE INVENTION

Although the rate of cancer incidence has declined since 1990, the number of people in the U.S. who are expected to die in 2004 from cancer is still expected to exceed half a million. The five most prevalent types of cancer in the U.S., ranked by the estimated number of new cases for the year 2004 (excluding base and squamous cell cancers of the skin), are as follows: prostate, female breast, lung and bronchus, colon and rectum, and urinary bladder. Breast cancer is the leading cause of cancer in U.S. women, with approximately 216,000 new cases diagnosed and 40,000 deaths per year.

Several modalities, including radiation, chemotherapy, and surgery, either alone or in combination, are being used for the treatment of cancer. Because of these treatments, most patients with skin cancer, and about half the people treated for internal cancers, are completely freed of their disease. However, the therapies now available for internal cancers often give rise to side effects so harmful that they compromise the benefits of treatment, and existing therapies for such internal cancers often fail in many cases. Radiation and surgery are limited in that they cannot treat widespread metastases that eventually form full-fledged tumors at numerous sites. In the 1960's it was discovered that chemotherapy could cure some cancers when several drugs were given in combination. Unfortunately, the most common cancers (breast, lung, colorectal, and prostate cancer) are not yet curable with chemotherapy alone.

One way to make treatments for cancer more specific would be to utilize the receptors that are overexpressed on the surface of cancer cells. There has been a great deal of research in recent years that has provided much information about these receptors, some of which is summarized herein below.

Urokinase-type plasminogen activator (uPA) receptor: uPA, also known as urokinase, appears to be the enzyme primarily responsible for the generation of plasmin during the process of extracellular matrix degradation. The ability of cancer cells to degrade extracellular matrices is critical to the metastasis of these cells. In all types of human cancers studied so far, both uPA and uPA receptors are consistently found to be present at the invasive foci of the tumors (Ellis et al., 1992). uPA consists of an A chain and a B chain, with the A chain responsible for binding to the receptor (Stopelli et al., 1985). Further studies have shown that residues 12-32 in the A chain (SEQ ID NO: 11) are critical for binding to the receptor (Appella et al., 1987).

Epidermal growth factor (EGF) receptor: Transforming growth factor-α, with a molecular weight of 6 kDa, binds to this receptor with about the same affinity as EGF for mammalian cells (Marquardt et al., 1984). Human cancer cells often express high levels of this receptor (Phillips et al., 1994; and Pastan et al., 1992). This receptor has been targeted by a fusion protein consisting of the binding peptide linked to Pseudomonas exotoxin with its binding domain removed (Phillips et al., 1994). The problem with this approach is that normal cells with the receptors bound by the fusion protein are also killed, resulting in potentially severe side effects. For example, there are high concentrations of EGF receptors in the human liver.

Insulin-like growth factor I (IGF-I) receptor: IGF-I, also known as somatomedin C, is a 70-amino acid peptide and is a member of a family of structurally related peptides that includes insulin and IGF-II (Prior et al., 1991). The insulin-like growth factor I receptor (IGF-IR) is a ubiquitous and multifunctional tyrosine kinase that has been implicated in breast cancer development. In estrogen receptor-positive breast tumors, the levels of IGF-I are often elevated (Bartucci et al., 2001).

Interleukin-4 (IL-4) receptor: IL-4 is a 20,000 kDa protein produced by activated T lymphocytes and was first described as a growth factor for B lymphocytes (Howard et al., 1982). The IL-4 receptor is expressed by several types of cancer cells, including those of the breast. IL-4 has been shown to inhibit the growth of, and induce apoptosis (programmed cell death) in breast cancer cells (Gooch et al., 1998).

Interleukin-6 (IL-6) receptor: IL-6, with a molecular weight of 20,000, has been shown to act directly on activated B cells to induce immunoglobulin production (Muraguchi et al., 1988). Certain breast cancer cells express high affinity IL-6 receptors. Proliferation of breast cancer cells with iL-6 receptors has been shown to be inhibited by IL-6 (Chen et al., 1991).

Results obtained over the past 40 years have demonstrated that tumor cells of all types tested have an elevated growth requirement for methionine compared to normal cells (Miki et al., 2000). Numerous lines of cancer cells are unable to survive and grow when the amino acid methionine is replaced in the medium with homocystine. However, normal adult cell lines survive and grow well with this substitution. For example, Halpern et al. (1974) showed that breast carcinosarcoma and lymphatic leukemia cells did not retain viability after 20 days in media devoid of methionine but with added homocystine. On the other hand, normal liver fibroblasts, breast fibroblasts, and prostate fibroblasts grew normally under these same conditions. Further studies have shown that methionine-dependent cells arrest in the G₂ and G₁ phases of the cell cycle and subsequently die at methionine concentrations less than 5 μM regardless of high concentrations of homocystine precursors and folates (Kokkinakis et al., 1997a).

Subsequent to the tests of the effect of methionine on cancer and normal cells in cell culture, there have been in vivo tests of the effect of methionine depletion on cancer cells. One comprehensive study was performed on mice with human brain tumor xenografts (Kokkinakis et al., 1997b). With a combination of dietary restriction of methionine, homocysteine, and choline and synchronous treatments with intraperitoneal injections of L-methioninase (44 mg/kg per day of L-methioninase) and homocystine, tumor stasis was achieved in 100% of treated animals within four days of treatment, and regression was seen in one-third of animals after a 10-day period. The methioninase produced no toxicity in the mice.

However, the current methioninase experimental methodologies require large dosages of methioninase as well as methionine-, homocystine-, and choline-restricted diets. There is currently no method for targeting anticancer agents such as methioninase specifically to the surface of cancer cells, or specifically to the surface of blood vessels supplying the cancer cells. It is to such methods of targeting anticancer agents to the surface of cancer cells or blood vessels supplying the cancer cells, thereby requiring significantly lower dosages of anticancer agents than current methods and eliminating the need for dietary restrictions, and thus overcoming the disadvantages and defects of the prior art, that the present invention is directed.

### BRIEF DESCRIPTIONS OF THE DRAWINGS

This patent or application file contains at least one drawing executed in color. Copies of this patent or patent application publication with color drawing(s) will be provided by the Office upon request and payment of the necessary fee.

FIG. 1 is an SDS-PAGE analysis with Coomassie blue staining of the expression and purification of the fusion protein consisting of the first 49 amino acids of the urokinase A chain coupled to L-methioninase (designated “ATF-methioninase”; position indicated by the arrow). The fusion protein was expressed from plasmid pKK223-3 under control of the tac promoter in E. coli JM105 at 37° C. Lanes: 1, soluble lysate; 2, heat treated soluble lysate; 3, pooled fractions from anion exchange chromatography at pH 8.0; 4, pooled fractions from hydrophobic interaction chromatography; 5, pooled fractions from anion exchange chromatography at pH 6.5; M, molecular weight markers are indicated on the right in kDa.

FIG. 2 illustrates the effects of methionine deficiency on MCF-7 cell migration. Each bar represents the mean distance of cell migration into the wounded area from 10-12 microscopic fields±SEM. Met+ indicates a methionine concentration of 15 mg/l; Hcy+ indicates a homocystine concentration of 15 mg/l; Met− and Hcy− indicate an absence of methionine and homocystine, respectively, in the media.

FIG. 3 illustrates the effects of methionine deficiency on MCF-7 cell proliferation index. Each bar represents the mean cell number in the wounded area from 10-12 microscopic fields±SEM. Met+ indicates a methionine concentration of 15 mg/l; Hcy+ indicates a homocystine concentration of 15 mg/l; Met− and Hcy− indicate an absence of methionine and homocystine, respectively, in the media.

FIG. 4 illustrates the dose-response effect of the ATF-methioninase fusion protein on MCF-7 cell migration. Each bar represents the mean distance of cell migration into the wounded area from 10-12 microscopic fields±SEM.

FIG. 5 illustrates a dose-response effect of the ATF-methioninase fusion protein on MCF-7 cell proliferation index. Each bar represents the mean cell number in the wounded area from 10-12 microscopic fields±SEM.

FIG. 6 illustrates urokinase-induced displacement of the ATF-methioninase fusion protein from membrane binding sites in MCF-7 cell. The data presented in this figure is summarized from two experiments. The concentration or human urokinase that produced a 50% displacement of fusion protein is shown by the dotted line.

FIG. 7 illustrates the weight of nude mice during the treatment period with ATF-methioninase fusion protein or vehicle control. Mice were injected with MCF-7 human breast cancer cells 30 days before treatment started.

FIG. 8 illustrates tumor volume change following 2-week treatment period of nude mice with ATF-methioninase fusion protein or vehicle control. Mice were injected with MCF-7 human breast cancer cells 30 days before treatment started.

FIG. 9 illustrates the total number of cancer cells per weight of tissue after 2-week treatment of nude mice with ATF-methioninase fusion protein or vehicle control. Mice were injected with MCF-7 human breast cancer cells 30 days before treatment started.

FIG. 10 illustrates SDS-PAGE analysis with Coomassie blue staining of the expression and purification of ATF-methioninase fusion protein (position indicated by the arrow). The fusion protein was expressed from plasmid pET-30/Ek/LIC/ATF-Meth in E. coli BL21(DE3) cells at 30° C. (lane 1, whole cells; lane 2, soluble lysate; lane 3, eluted fraction from first metal affinity chromatography; lane 4, eluted fraction after cleavage with HRV 3C protease; lane 5, pooled fractions from second metal affinity chromatography; M, marker proteins with molecular masses indicated on the left in kiloDaltons).

FIG. 11 illustrates the dose-response effect of ATF-methioninase fusion protein on MCF-7 breast cancer cell proliferation. Fusion protein (FP); mutated FP (M-FP); L-methioninase (L-M) were administered immediately following culture wounding. Each bar represents the number of cells that migrated into the wounded area (mean±SEM from 10 to 12 microscope fields).

FIG. 12 illustrates the dose-response effect of ATF-methioninase fusion protein on MCF-7 breast cancer cell migration. Fusion protein (FP); mutated fusion protein (M-FP); L-methioninase (L-M) were administered immediately following culture wounding. Each bar represents the distance of cell migration into the wounded area (mean±SEM from 10 to 12 microscope fields).

FIG. 13 illustrates the dose-response effect of ATF-methioninase fusion protein on PC-3 prostate cancer cell proliferation. Fusion protein (FP); mutated FP (Mute-FP); L-methioninase (L-M) were administered immediately following culture wounding. Each bar represents the number of cells that migrated into the wounded area (mean±SEM from 10 to 12 microscope fields).

FIG. 14 illustrates the dose-response effect of ATF-methioninase fusion protein on PC-3 prostate cancer cell migration. Fusion protein (FP); mutated fusion protein (Mute-FP); L-methioninase (L-M) were administered immediately following culture wounding. Each bar represents the distance of cell migration into the wounded area (mean±SEM from 10 to 12 microscope fields).

FIG. 15 illustrates the dose-response effect of ATF-methioninase fusion protein on SK-LU-1 lung cancer cell proliferation. Fusion protein (FP); mutated FP (M-FP); L-methioninase (L-M) were administered immediately following culture wounding. Each bar represents the number of cells that migrated into the wounded area (mean±SEM from 10 to 12 microscope fields)

FIG. 16 illustrates the dose-response effect of ATF-methioninase fusion protein on SK-LU-1 lung cancer cell migration. Fusion protein (FP); mutated FP (M-FP); L-methioninase (L-M) were administered immediately following culture wounding. Each bar represents the distance of cell migration into the wounded area (mean±SEM from 10 to 12 microscope fields).

FIG. 17 illustrates the dose-response effect of TGF-methioninase fusion protein on MCF-7 breast cancer cell proliferation. Fusion protein (TGF) was administered immediately following culture wounding. Each bar represents the number of cells that migrated into the wounded area (mean±SEM from 10 to 12 microscope fields).

FIG. 18 illustrates the dose-response effect of TGF-methioninase fusion protein on MCF-7 breast cancer cell migration. Fusion protein (TGF) was administered immediately following culture wounding. Each bar represents the distance of cell migration into the wounded area (mean±SEM from 10 to 12 microscope fields).

FIG. 19 illustrates photomicrographs (40×) of MCF-7 cells treated for 18 hours with either: vehicle control (C); ATF-methioninase fusion protein (FP); mutated ATF-methioninase fusion protein (M-FP); L-methioninase (L-M); or ATF-methioninase fusion protein+urokinase (UK+FP). All treatments were for 18 hours at a concentration of 10−6 M. The red/brown color represents positive staining of the L-methioninase-specific primary antibody.

FIG. 20 illustrates the effect of a 20-day ATF-methioninase fusion protein treatment on the growth of MCF-7 tumors and in nude mouse xenografts. The cell number/mg of tissue was quantified by measuring β-gal activity in tissue homogenates. Ten animals were included in each group. Treatment was administered by intra-tumoral injection.

FIG. 21 illustrates a scheme for the purification of proteins using a HisTrap column with immobilized nickel.

FIG. 22 illustrates an SDS-PAGE analysis with Coomassie blue staining of three of the purified proteins. Lane 1, annexin V; lane 2, L-methioninase; lane 3, methioninase-annexin V; M, marker proteins with molecular masses indicated on the left in kiloDaltons.

FIG. 23 illustrates the binding of methioninase-annexin V fusion protein (FP) to phosphatidylserine adsorbed to plastic. Blank for assay: −FP, +1° Ab, +2° Ab. Control for assay (▴): +L-methioninase, +1° Ab, +2° Ab (each A₄₅₀ measured in triplicate).

FIG. 24 illustrates the binding of methioninase-annexin V fusion protein (FP) to phosphatidylserine exposed on MCF-7 breast cancer cells. Blank for assay: —FP, +1° Ab, +20 Ab. Control for assay (▴): +L-methioninase, +10 Ab, +20 Ab (each A₄₅₀ measured in triplicate).

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

According to the present invention, conjugates for use in the treatment for cancer are provided. The present invention provides conjugates that include a ligand having the ability to specifically and stably bind to an external receptor or binding site on an outer surface of a tumor vasculature endothelial cell or cancer cell, wherein the external receptor or binding site is specific for tumor vasculature endothelial cells or cancer cells (i.e., is uniquely expressed or overexpressed on a luminal surface of the tumor vasculature endothelial cell or cancer cell); the conjugate is maintained on the outer surface of the tumor vasculature endothelial cell or cancer cell with substantially no internalization of the conjugate. The conjugate further includes an anticancer agent that is operatively attached to the ligand, wherein the anticancer agent is selectively toxic to cancer cells. The ligand portion of the conjugate specifically and stably binds to the external receptor or binding site on the outer surface of the cell and is maintained on the surface of the cell with substantially no internalization. The ligand may be selected from the group consisting of urokinase, epidermal growth factor (EGF), transforming growth factor-alpha (TGFα), insulin-like growth factor, interleukin-4 (IL-4), interleukin-6 (IL-6), platelet-derived growth factor (PDGF), fibroblast growth factor (FGF), laminin, vascular endothelial growth factor (VEGF), annexin V, antibodies to a receptor or aminophospholipid that is uniquely expressed or overexpressed on a surface of a tumor vasculature endothelial cell or cancer cell, and fragments or variants thereof which substantially retain the ability to bind to the receptor or binding site. The anticancer agent may be selected from the group consisting of L-methioninase and fragments and variants thereof which substantially retain the ability to degrade methionine, and L-asparaginase and fragments and variants thereof which substantially retain the ability to degrade asparagine. The anticancer agent and the ligand may be directly coupled together or indirectly coupled together via a linker. In addition, the anticancer agent may be conjugated to PEG, or the conjugate may be encapsulated in a liposome.

In one embodiment, the receptor or binding site to which the ligand binds may be an aminophospholipid such as, but not limited to, phosphatidylserine or phosphatidylethanolamine. In such embodiment, the ligand of the conjugate would comprise an aminophospholipid binding domain. Non-limiting examples of proteins and peptides that comprise an aminophospholipid binding domain that may function as the ligand of the conjugate in accordance with the present invention are described in greater detail herein below.

In one embodiment, the conjugate has an amino acid sequence comprising at least one of: (A) an amino acid sequence essentially as set forth in any of SEQ ID NOS:1, 15 and 23; (B) an amino acid sequence encoded by any of SEQ ID NOS:2, 16 and 24; (C) an amino acid sequence that is substantially identical to (A) or (B); (D) an amino acid sequence that is a variant of (A) or (B); and (E) an amino acid sequence that is a fragment of (A) or (B).

The present invention also includes a purified nucleic acid segment encoding the conjugate described herein above, a recombinant vector comprising such a purified nucleic acid segment, and a recombinant host cell comprising the recombinant vector.

The present invention further includes a pharmaceutical composition that comprises a pharmaceutically acceptable carrier, such as, but not limited to PEG, liposomes, ethanol, DMSO, aqueous buffers, oils, and combinations thereof, and a therapeutically effective amount of the conjugate described herein above.

The present invention further includes methods of treating a cancer tumor or cancer cells supplied by a tumor vasculature. In one embodiment, the method includes providing the conjugate described herein above and contacting at least one blood vessel supplying a tumor with a therapeutically effective amount of the conjugate, whereby the conjugate is maintained on the outer surface of the tumor vasculature endothelial cell with substantially no internalization of the conjugate, and wherein the L-methioninase is stably bound to the outer surface of the tumor vasculature endothelial cell such that exogenous methionine in a vicinity or close proximity to the tumor vasculature endothelial cell is sufficiently depleted (i.e., degraded) and thus, not delivered via the tumor vasculature to the cancer tumor or cancer cells, whereby the conjugate is selectively toxic to cancer cells being supplied by the at least one blood vessel. The L-methioninase is stably bound to the outer surface of the tumor vasculature endothelial cell such that the methionine coming in contact with the endothelial cell surface is degraded and thus, not allowed to cross the endothelial cell surface to nourish the cancer cells served by the tumor vasculature, and therefore, the cancer cells die because of a lack of methionine.

In another embodiment, the conjugate described herein above is provided, and a population of tumor cells is contacted with a therapeutically effective amount of the conjugate such that the conjugate is specifically and stably bound to an outer surface of the cancer cell with substantially no internalization thereof. In this manner, exogenous methionine in a vicinity of the cancer cell is sufficiently depleted, whereby the conjugate is selectively toxic to cancer cells.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo-, or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed.), Cold Spring Harbor Laboratory Press, Cold Spring Harbor, N.Y. (1989) and Ausubel et al. Current Protocols in Molecular Biology (Wiley Interscience (1988)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of animals.

As utilized in accordance with the present disclosure, the following terms, unless otherwise indicated, shall be understood to have the following meanings:

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a coding sequence isolated away from, or purified free from, unrelated genomic DNA, genes and other coding segments. Included within the term “DNA segment”, are DNA segments and smaller fragments of such segments, and also recombinant vectors, including, for example, plasmids, cosmids, phage, viruses, and the like. In this respect, the term “gene” is used for simplicity to refer to a functional protein-, polypeptide-, or peptide-encoding unit. As will be understood by those in the art, this functional term includes genomic sequences, cDNA sequences or combinations thereof. “Isolated substantially away from other coding sequences” means that the gene of interest forms the significant part of the coding region of the DNA segment, and that the DNA segment does not contain other non-relevant large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

Preferably, DNA sequences in accordance with the present invention will further include genetic control regions which allow the expression of the sequence in a selected recombinant host. The genetic control region may be native to the cell from which the gene was isolated, or may be native to the recombinant host cell, or may be an exogenous segment that is compatible with and recognized by the transcriptional machinery of the selected recombinant host cell. Of course, the nature of the control region employed will generally vary depending on the particular use (e.g., cloning host) envisioned.

Truncated genes also fall within the definition of preferred DNA sequences as set forth above. Those of ordinary skill in the art would appreciate that simple amino acid removal can be accomplished, and the truncated versions of the sequence simply have to be checked for the desired biological activity in order to determine if such a truncated sequence is still capable of functioning as required. In certain instances, it may be desired to truncate a gene encoding a protein to remove an undesired biological activity, as described herein.

Nucleic acid segments having a desired biological activity may be isolated by the methods described herein. The term “a sequence essentially as set forth in SEQ ID NO:X” means that the sequence substantially corresponds to a portion of SEQ ID NO:X and has relatively few amino acids or codons encoding amino acids which are not identical to, or a biologically functional equivalent of, the amino acids or codons encoding amino acids of SEQ ID NO:X. The term “biologically functional equivalent” is well understood in the art and is further defined in detail herein, as a gene having a sequence essentially as set forth in SEQ ID NO:X, and that is associated with the ability to perform a desired biological activity in vitro or in vivo.

The art is replete with examples of practitioner's ability to make structural changes to a nucleic acid segment (i.e., encoding conserved or semi-conserved amino acid substitutions) and still preserve its enzymatic or functional activity when expressed. See for special example of literature attesting to such: (1) Risler et al., “Amino Acid Substitutions in Structurally Related Proteins. A Pattern Recognition Approach.” J. Mol. Biol. 204:1019-1029 (1988) [“ . . . according to the observed exchangeability of amino acid side chains, only four groups could be delineated; (i) Ile and Val; (ii) Leu and Met, (iii) Lys, Arg, and Gln, and (iv) Tyr and Phe.”]; (2) Niefind et al., “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al., “Environment-Specific Amino Acid Substitution Tables: Tertiary Templates and Prediction of Protein Folds,” Protein Science 1:216-226 (1992) [“Analysis of the pattern of observed substitutions as a function of local environment shows that there are distinct patterns . . . . Compatible changes can be made.”]

These references and countless others, indicate that one of ordinary skill in the art, given a nucleic acid sequence or an amino acid or an amino acid sequence, could make substitutions and changes to the nucleic acid sequence without changing its functionality. One of ordinary skill in the art, given the present specification, would be able to identify, isolate, create, and test DNA sequences and/or enzymes that produce natural or chimeric or hybrid molecules having a desired biological activity. As such, the presently claimed and disclosed invention should not be regarded as being solely limited to the specific sequences disclosed herein. Standardized and accepted functionally equivalent amino acid substitutions are presented in Table I.

TABLE I

| | Conservative and Semi- |
|---|---|
| Amino Acid Group | Conservative Substitutions |
| Nonpolar R Groups | Alanine, Valine, Leucine, Isoleucine, |
| | Proline, Methionine, Phenylalanine, |
| | Tryptophan |
| Polar, but uncharged, R Groups | Glycine, Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

The DNA segments of the present invention encompass DNA segments encoding biologically functional equivalent proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to antigenicity of the protein or to test mutants in order to examine biological activity at the molecular level or to produce mutants having changed or novel enzymatic activity and/or substrate specificity.

By “polypeptide” is meant a molecule comprising a series of amino acids linked through amide linkages along the alpha carbon backbone. Modifications of the peptide side chains may be present, along with glycosylations, hydroxylations and the like. Additionally, other nonpeptide molecules, including lipids and small molecule agents, may be attached to the polypeptide.

Another preferred embodiment of the present invention is a purified nucleic acid segment that encodes a protein in accordance with the present invention, further defined as being contained within a recombinant vector. As used herein, the term “recombinant vector” refers to a vector that has been modified to contain a nucleic acid segment that encodes a desired protein or fragment thereof. The recombinant vector may be further defined as an expression vector comprising a promoter operatively linked to said nucleic acid segment.

A further preferred embodiment of the present invention is a host cell, made recombinant with a recombinant vector comprising one or more genes encoding one or more desired proteins, such as a conjugate. The preferred recombinant host cell may be a prokaryotic cell. In another embodiment, the recombinant host cell is an eukaryotic cell. As used herein, the term “engineered” or “recombinant” cell is intended to refer to a cell into which one or more recombinant genes have been introduced mechanically or by the hand of man. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter associated, or not naturally associated, with the particular introduced gene.

In preferred embodiments, the DNA segments further include DNA sequences, known in the art functionally as origins of replication or “replicons”, which allow replication of contiguous sequences by the particular host. Such origins allow the preparation of extrachromosomally localized and replicating chimeric or hybrid segments of plasmids, to which the desired DNA sequences are ligated. In more preferred instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The nucleic acid segments of the present invention, regardless of the length of the coding sequence itself, may be combined with other DNA sequences, such as promoters, polyadenylation signals, additional restriction enzyme sites, multiple cloning sites, epitope tags, polyhistidine regions, other coding segments, and the like, such that their overall length may vary considerably. It is, therefore, contemplated that a nucleic acid fragment of almost any length may be employed, with the total length preferably being limited by the ease of preparation and use in the intended recombinant DNA protocol.

As used herein, a “conjugate” refers to a molecule that contains at least one receptor-binding ligand and at least one anticancer agent that are coupled directly or via a linker and that are produced by chemical coupling methods or by recombinant expression of chimeric DNA molecules to produce fusion proteins.

As used herein, the term “covalently coupled”, “linked”, “bonded”, “joined”, and the like, with reference to the ligand and anticancer agent components of the conjugates of the present invention, mean that the specified components are either directly covalently bonded to one another or indirectly covalently bonded to one another through an intervening moiety or components, such as a bridge, spacer, linker or the like. For example but not by way of limitation, the ligand and the anticancer agent may be chemically coupled together via a thioether linkage as described in Mickisch et al. (1993).

As used herein, the term “anticancer agent” refers to a molecule capable of inhibiting cancer cell function. The agent may inhibit proliferation or may be cytotoxic to cells. A variety of anticancer agents can be used, and include those that inhibit protein synthesis and those that inhibit expression of certain genes essential for cellular growth or survival. Anticancer agents include those that result in cell death and those that inhibit cell growth, proliferation and/or differentiation. Preferably, the anticancer agent is selectively toxic against certain types of cancer cells but does not affect or is less effective against other normal cells. For example, but not by way of limitation, the anticancer agent may be a protein which degrades a nonessential amino acid wherein the nonessential amino acid is still required for growth of tumor cells, such as, but not limited to, methioninase and asparaginase. In another embodiment, the anticancer agent is an antineoplastic agent.

The term “antineoplastic agent” is used herein to refer to agents that have the functional property of inhibiting a development or progression of a neoplasm in a human or animal, particularly a malignant (cancerous) lesion, such as a carcinoma, sarcoma, lymphoma, or leukemia. Inhibition of metastasis is frequently a property of antineoplastic agents.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example, but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease in conjunction with the conjugates of the present invention. This concurrent therapy can be sequential therapy where the patient is treated first with one drug, and then the other, or the two drugs are given simultaneously.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis, it is more abundant than any other individual species in the composition), and preferably, a substantially purified fraction is a composition wherein the object species comprises at least about 50% (on a molar basis) of all macromolecular species present. Generally, a substantially pure composition will comprise more than about 80% of all macromolecular species present in the composition, more preferably, more than about 85%, 90%, 95%, and 99%. Most preferably, the object species is purified to essential homogeneity (contaminant species cannot be detected in the composition by conventional detection methods), wherein the composition consists essentially of a single macromolecular species.

A “liposome” is a small vesicle composed of various types of lipids, phospholipids and/or surfactant. The components of the liposome are commonly arranged in a bilayer formation, similar to the lipid arrangement of biological membranes.

The terms “cancer” and “cancerous” refer to, or describe, the physiological condition in mammals that is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

The term patient includes human and veterinary subjects. “Mammal”, for purposes of treatment, refers to any animal classified as a mammal, including human, domestic and farm animals, nonhuman primates, and any other animal that has mammary tissue.

The terms “treat”, “treating” and “treatment”, as used herein, will be understood to include both inhibition of tumor growth, as well as induction of tumor cell death.

The term “receptor”, as used herein, will be understood to include any peptide, protein, glycoprotein, polycarbohydrate, or lipid that is uniquely expressed or overexpressed on the surface of cancer cells or cells in the tumor vasculature and is exposed on the surface of cancer cells or cells in the tumor vasculature in a manner that will allow interaction with a circulating targeting agent, such as the conjugate.

The ligand of the conjugate of the present invention may be any protein or composition which binds to the receptor or other targeting molecule uniquely present on the surface of cancer cells or cells in the tumor vasculature (i.e., an aminophospholipid). When the ligand is a protein, the ligand may contain the entire protein that binds to the desired receptor or other targeting molecule, or the ligand may contain only a portion of the protein. For example, it may be desirable to remove a portion of the protein that has an undesirable biological activity, or it may be desirable to remove a portion of the protein to enable attachment of the anticancer agent. The only requirement when a portion of the protein is present as the ligand in the conjugate, is that the portion of the protein substantially retain the protein's receptor or targeting molecule binding activity. In addition, if the protein contains a portion that targets the protein for internalization, such portion should be removed so that the conjugate of the present invention is stably bound to the outer surface of the cancer cell or blood vessel supplying the tumor and is maintained thereon with substantially no internalization thereof. The terms “portion” and “fragment” are used herein interchangeably.

Likewise, the conjugate may contain a variant of the ligand. For example, it may be desirable to modify a portion of the ligand that has an undesirable biological activity, or it may be desirable to modify a portion of the ligand to enable attachment of the anticancer agent. The only requirement when a variant of the ligand is present in the conjugate, is that the ligand variant substantially retain the ligand's receptor or targeting molecule binding activity. Also, sequences may be added to, or inserted within, the ligand during modification, as long as the modified ligand substantially retains the ligand's receptor binding activity. Therefore, it is to be understood that the term “ligand variant” includes both substitutions (including but not limited to conservative and semi-conservative substitutions) as well as additions and insertions to the native ligand's sequence that do not substantially affect the ligand's receptor binding activity. Such variations may occur at the nucleic acid level during construction of the construct from which the conjugate is expressed, or the variations may be produced by other posttranscriptional or posttranslational means known to those or ordinary skill in the art, including but not limited to, mutations and chemical modifications.

Examples of receptors that may be targeted by conjugates in accordance with the present invention include urokinase receptor, epidermal growth factor (EGF) receptor, insulin-like growth factor receptor, interleukin-4 (IL-4) receptor, interleukin-6 (IL-6) receptor, keratinocyte growth factor (KGF) receptor, platelet-derived growth factor (PDGF) receptor, fibroblast growth factor (FGF) receptor, laminin receptor, vascular endothelial growth factor (VEGF) receptor, transferrin receptor, phosphatidylserine (PS), phosphatidylethanolamine (PE), fibronectin, and the like, as well as portions thereof, and variants thereof, that substantially maintain the ability to bind to the ligand of the conjugate of the present invention and maintain the conjugate on the surface of the cell with substantially no internalization thereof.

The conjugate may contain all, or a portion or variant, of one of the following ligands to target the conjugate to one or more of the above receptors: urokinase, epidermal growth factor (EGF), transforming growth factor-alpha (TGFα), insulin-like growth factor, interleukin-4 (IL-4), interleukin-6 (IL-6), platelet-derived growth factor (PDGF), fibroblast growth factor (FGF), laminin, vascular endothelial growth factor (VEGF), annexin V, antibodies or antibody fragments (such as, but not limited to, antibodies to the transferrin receptor or the ED-B domain of fibronectin), and the like. The structure and properties of some of the above-listed growth factors are very similar, and therefore, one growth factor may be utilized to target another receptor (for example, TGFα may be utilized to bind to the EGF receptor).

There are numerous reports in the literature of antibodies or antibody fragments binding to cancer cells or cells supplying tumor vasculature. Examples of such binding can be found in Batra et al. (1989), wherein a monoclonal antibody was chemically coupled to Pseudomonas exotoxin, and Nilsson et al. (2001), wherein a single chain antibody fragment, denoted scFv, was fused to a peptide linker in a recombinant protein to soluble tissue factor. Examples of antibodies binding to cells of the tumor vasculature are described in more detail herein below.

Aminophospholipids are largely absent from the surfaces of resting mammalian cells under normal conditions. Phosphatidylserine (PS), an anionic phospholipid, is the most abundant aminophospholipid of the plasma membrane and is tightly segregated to the internal side of the plasma membrane in most cell types. Recently, it has been discovered that PS and phosphatidylethanolamine (PE), a neutral or zwitterionic phospholipid, are expressed on the outside surface of the endothelial cells that line the blood vessels in tumors in mice but are not expressed on the outside surface of the vascular endothelium in normal organs (Ran et al., 2002a and 2002b). In addition, PS and PE have been shown to be expressed on the outside surface (i.e., luminal surface) of cancer cells (Sugimura et al., 1994; and Rao et al., 1992).

An “aminophospholipid”, as used herein, means a phospholipid that includes within its structure at least a first primary amino group. Preferably, the term “aminophospholipid” is used to refer to a primary amino group-containing phospholipid that occurs naturally in mammalian cell membranes. However, this is not a limitation on the meaning of the term “aminophospholipid”, as this term also extends to non-naturally occurring or synthetic aminophospholipids that nonetheless have uses in the invention, e.g., as an immunogen in the generation of anti-aminophospholipid antibodies (“cross-reactive antibodies”) that do bind to aminophospholipids of mammalian plasma membranes. The aminophospholipids of U.S. Pat. No. 5,767,298, incorporated herein by reference, are appropriate examples.

Annexin V, a human protein that is a member of the annexin family of calcium-dependent phospholipid-binding proteins, binds with very high affinity to PS-containing phospholipid bilayers, and therefore, such protein, or an effective aminophospholipid-binding portion thereof, may be utilized as the ligand portion of the conjugates of the present invention. Annexin V is a 35 kDa monomeric protein (SEQ ID NO:25, encoded by SEQ ID NO:26), which has been crystallized and shown to consist of four tandem repeats of similar structure (Huber et al., 1990). Structural evidence demonstrates that the N terminus of annexin V is located at the surface of the protein and faces away from the membrane-binding side of the molecule (Huber et al., 1990; Concha et al., 1993 and Voges et al., 1994). It was later found that the attachment of prourokinase at the N terminus of annexin V did not alter its affinity for cell membranes in which phosphatidylserine (PS) was exposed on the membrane surface (Tait et al., 1995), which is consistent with the previous structural evidence.

Other ligands which may be used in conjugation with anticancer agents as contemplated herein include, but are not limited to, RGD-motif peptides (Receptor: integrins alpha-v-beta 3 and alpha-v-beta 5); NGR-motif peptides (Receptor: aminopeptidase N, also known as CD13); F3, a 34-amino acid basic peptide from HMGN2 (Receptor: cell surface nucleolin) (Ruoslahti et al., 2004); HWGF (SEQ ID NO:27)-motif peptides (selective inhibitors of matrix metalloproteinase-2 and matrix metalloproteinase-9, also known as gelatinase A and gelatinase B (Koivunen et al., 1999)); the synthetic peptide CTTHWGFTLC (SEQ ID NO:28) (which targets antiogenic blood vessels, inhibits the migration of human endothelial cells and tumor cells, and also prevents tumor growth and invasion in animal models and improves survival of mice bearing human tumors) (Koivunen et al., 1999); and the amino-terminal fragment (ATF) of urokinase (amino acids 1-135 of urokinase A chain). ATF binds to the urokinase receptor, but, unlike full length urokinase, is not internalized (Xu et al., 1997).

Alternatively, the ligand of the conjugate of the present invention may be an aminophospholipid-specific antibody, such as a phosphatidylserine-specific or phosphatidylethanolamine-specific monoclonal antibody, to which the anticancer agent is conjugated. Examples of PS- and PE-specific monoclonal antibodies include those described in U.S. Pat. Nos. 6,312,694; 6,406,693; 6,783,760; 6,818,213; and 7,067,109. The ligand to which the anticancer agent is associated may be a non-PS-binding moiety which binds to another tumor-specific feature, such as those described in U.S. Pat. Nos. 6,451,312; 6,093,399; 6,004,555; and 6,051,230. The present invention contemplates other tumor/cancer-specific external receptors other than aminophospholipids as targets for the conjugates of the present invention. Such receptors include, for example, those described in U.S. Pat. Nos. 6,818,213; 6,783,760; 6,451,312; and 6,406,693. All of the patents, published applications and publications listed herein are hereby expressly incorporated herein by reference in their entireties.

The modification of one of the receptor-binding ligands described herein above to provide a fragment or variant thereof that substantially maintains the receptor-binding ability of the native receptor-binding ligand is fully within the skill of a person in the art and therefore is also within the scope of the present invention. The term “substantially maintains the receptor-binding ability of the native receptor-binding ligand” means that the protein fragment or variant maintains at least 50% of the native ligand's receptor-binding ability, and preferably, at least 75% of the native ligand's receptor-binding ability, and more preferably, at least 90% of the native ligand's receptor-binding ability.

The phrase “substantially no internalization”, as used herein, refers to a lack of internalization of a substantial amount of the conjugates of the present invention. For example, the phrase “substantially no internalization” will be understood as less than 25% of the conjugates of the present invention being internalized by a cell to which the conjugate is bound, or less than 10% of the conjugates of the present invention being internalized by a cell to which the conjugate is bound, or less than 5% of the conjugates of the present invention being internalized by a cell to which the conjugate is bound, or less than 3% of the conjugates of the present invention being internalized by a cell to which the conjugate is bound, or less than 1% of the conjugates of the present invention being internalized by a cell to which the conjugate is bound.

The anticancer agent is preferably an enzyme that is selectively toxic to cancer cells and does not affect normal cells. In this manner, the technology of the present invention will selectively target the anticancer agent to receptors on the surface of cancer cells or cells in the tumor vasculature in order to stop the growth of the cancer cells, thus leading to a more effective treatment to eliminate cancers. For example, the anticancer agent may be a protein which degrades a nonessential amino acid wherein the nonessential amino acid is still required for growth of tumor cells, such as, but not limited to, L-methioninase and L-asparaginase.

One example of an anticancer agent that may be utilized in accordance with the present invention is L-methioninase. Cancer cells of all types have an elevated requirement for methionine compared to normal cells, and all exogenous methionine in the vicinity of the cancer cells will be substantially depleted with L-methioninase bound to the cell surface in accordance with the present invention. The use of L-methioninase as an antitumor reagent in anti-methionine chemotherapy has been well documented and is described in detail in U.S. Pat. No. 5,690,929, issued to Lishko et al. on Nov. 25, 1997; U.S. Pat. No. 5,888,506, issued to Tan on Mar. 30, 1999; and U.S. Pat. No. 6,231,854, issued to Yuying on May 15, 2001, the contents of each of which are hereby expressly incorporated herein by reference in their entirety.

Purified L-methioninase from any source may be utilized in accordance with the present invention. Optionally, recombinant L-methioninase expressed from any genes known in the art or later identified that have common activity and/or sequence identity with currently known L-methioninase sequences may be utilized in accordance with the present invention. Further, the L-methioninase utilized in accordance with the present invention may be truncated or modified to contain substitutions or insertions when compared with known L-methioninase sequences. The truncation or modification of L-methioninase sequences to provide a protein which substantially retains the ability to degrade methionine is fully within the skill of a person in the art and therefore, is also within the scope of the present invention.

The gene for L-methioninase from P. putida has been cloned by two different research groups (Hori et al., Cancer Res., 56:2116-2122 (1996), and Inoue et al., Biochem (Tokyo), 117:1120-1125 (1995)). The genes for two L-methioninases from the primitive protozoan parasite Trichomonas vaginalis have been cloned, and the two L-methioninases have been expressed in E. coli as a fusion with a six-histidine tag and were purified (McKie et al., J Biol. Chem., 273:5549-5556 (1998). The six-histidine tag was at the N-terminus for one of the L-methioninases and at the C-terminus for the other. Both of these recombinant fusion proteins produced very high methioninase activity.

Another example of an anticancer agent that may be utilized in accordance with the present invention is L-asparaginase. The use of L-asparaginase as an antitumor reagent in anti-asparagine chemotherapy has been well documented, and purification of L-asparaginase for use in chemotherapy has been described in U.S. Pat. No. 4,473,646, issued to Guy et al., on Sep. 25, 1984, the contents of which are hereby expressly incorporated herein by reference in their entirety. In addition, L-asparaginase has been approved for treatment of patients with acute lymphoblastic leukemia.

Purified L-asparaginase from any source may be utilized in accordance with the present invention. Optionally, recombinant L-asparaginase expressed from any genes known in the art or later identified that have common activity and/or sequence identity with currently known L-asparaginase sequences may be utilized in accordance with the present invention. Further, the L-asparaginase utilized in accordance with the present invention may be truncated or modified to contain substitutions or insertions when compared with known L-asparaginase sequences. The truncation or modification of L-asparaginase sequences to provide a protein which substantially retains the ability to degrade asparagine is fully within the skill of a person in the art and therefore, is also within the scope of the present invention.

Since the anticancer agents described herein are typically bacterially-derived proteins, the anticancer agent of the conjugate of the present invention may be modified so as to reduce the immunogenicity thereof. One method for reducing a protein's immunogenicity is to conjugate the protein to polyethylene glycol (PEG). L-methioninase has been successfully conjugated to PEG, resulting in a 36-fold increase in serum half-life and the elimination of immunogenic reactions while maintaining the same antitumor efficacy in vitro as the unmodified L-methioninase (Yang et al., 2004). In guinea pigs, there was no detectable immune response after L-methioninase conjugated to PEG was injected, while shock and death resulted when unmodified L-methioninase was injected. In a clinical trial of children with newly diagnosed acute lymphoblastic leukemia, L-asparaginase conjugated to PEG (pegasparaginase) was compared to native L-asparaginase (Avramis et al., Blood, 99:1986-1994 (2002)). In the first delayed intensification phase, 26% of the native L-asparaginase-treated patients had high-titer antibodies, whereas only 2% of the pegasparaginase-treated patients had those levels. In addition, the serum half-lives were 5.5 days for pegasparaginase and 26 hours for native L-asparaginase.

By “polyethylene glycol” or “PEG” is also meant any other polyalkylene glycol compound or a derivative thereof, with or without coupling agents or derivatization with coupling or activating moeities (e.g., with thiol, triflate, tresylate, azirdine, oxirane, or preferably with a maleimide moiety). Compounds, such as maleimido monomethoxy PEG, are exemplary or activated PEG compounds of the invention. Other polyalkylene glycol compounds, such as polypropylene glycol, may be used in the present invention. Other appropriate polymer conjugates include, but are not limited to, non-polypeptide polymers, charged or neutral polymers of the following types: dextran, colominic acids or other carbohydrate based polymers, biotin deriviatives and dendrimers, for example.

The term PEG is also meant to include other polymers of the class polyalkylene oxides. The PEG can be linked to any N-terminal amino acid of the conjugate, and/or can be linked to an amino acid residue downstream of the N-terminal amino acid, such as lysine, histidine, tryptophan, aspartic acid, glutamic acid, and cysteine, for example, or other such linkable amino acids known to those of skill in the art. Cysteine-pegylated conjugates, for example, are created by attaching polyethylene glycol to a thio group on a cysteine residue of the conjugate.

The PEG moiety attached to the conjugate may range in molecular weight, for example, from about 200 to 20,000 MW.

The conjugates contemplated herein can be adsorbed or linked to PEG molecules using techniques shown, for example (but not limited to), in U.S. Pat. Nos. 4,179,337; 5,382,657; 5,972,885; 6,177,087; 6,165,509; 5,766,897; and 6,217,869; and Published Application 2006/0275371; the specifications and drawings each of which are hereby expressly incorporated by reference herein in its entirety.

Another method for reducing a protein's immunogenicity is liposome encapsulation. In a study of L-asparaginase encapsulated in liposomes and administered to mice, the immune response was prevented, and the circulation time of the L-asparaginase was increased by a factor of up to 10 (Gaspar et al., Cancer Chemother Pharmacol., 38:373-377 (1996)).

Thus, the above-described studies demonstrate that the immunological response to the anticancer agent can be greatly reduced or eliminated by either conjugation to PEG or by encapsulation in liposomes, without significant effect on enzymatic activity of the anticancer agent. Liposome encapsulation has the advantage that covalent attachment of moieties to the enzyme is not required, which may be helpful to preserve binding of the proposed conjugates to the receptors on cancer cells.

The conjugate of the present invention may be administered to a subject by any methods known in the art, including but not limited to, oral, topical, transdermal, parenteral, subcutaneous, intranasal, intramuscular and intravenous routes, including both local and systemic applications. In addition, the conjugates of the present invention may be designed to provide delayed or controlled release using formulation techniques which are well known in the art.

The present invention also includes a pharmaceutical composition comprising a therapeutically effective amount of the conjugate described herein above in combination with a pharmaceutically acceptable carrier. As used herein, a “pharmaceutically acceptable carrier” is a pharmaceutically acceptable solvent, suspending agent or vehicle for delivering the conjugates of the present invention to the human or animal. The carrier may be liquid or solid and is selected with the planned manner of administration in mind. Examples of pharmaceutically acceptable carriers that may be utilized in accordance with the present invention include, but are not limited to, PEG, liposomes, ethanol, DMSO, aqueous buffers, oils, and combinations thereof.

The conjugate of the present invention provides several advantages of the methodologies of the prior art. First, since the anticancer agent is being targeted to cells that it is intended to kill, or the vasculature supplying the cells that it is intended to kill, the dosages of the conjugate containing the anticancer agent should be significantly lower than when the anticancer agent alone is administered systemically. Second, when the anticancer agent is L-methioninase, it may be possible to avoid having dietary restrictions of methionine, homocystine and choline, as in Kokkinakis et al. (1997b). Third, the interaction between the ligand of the conjugate and its respective receptor will displace the native ligand (such as urokinase or a growth factor) from the receptor, and, when the native ligand is involved in the invasive ability or biological advantage of the cancer cells, will greatly inhibit the proliferation and/or invasive ability of the cancer cells.

Examples are provided hereinbelow. However, the present invention is to be understood to not be limited in its application to the specific experimentation, results and laboratory procedures. Rather, the Examples are simply provided as one of various embodiments and are meant to be exemplary, not exhaustive.

### Example 1

Expression and Purification of ATF-methioninase. A pKK223-3 plasmid containing the gene for L-methioninase (containing 398 amino acids and with a calculated molecular weight of 42.7 kDa) from Pseudomonas putida was kindly provided by Dr. Dennis Carson of the University of California, San Diego (Hori et al., 1996). Plasmid pULB1221 containing the gene for human urokinase was kindly provided by Dr. Paul Jacobs of the Free University of Brussels, Belgium (Jacobs et al., 1985). Plasmid pKK223-3, with the tac promoter and an ampicillin resistance gene, was obtained from Amersham Biosciences (Piscataway, N.J.). E. coli JM105 was used as the host for both vector construction and protein expression.

The following fusion protein gene was constructed:

N-(amino acids 1-49 of urokinase A chain)-Gly-Ser-Gly-Ser-Gly-Ser-(L-methioninase)-C

The amino acid sequence of the fusion protein was assigned SEQ ID NO:1, while the nucleic acid sequence of the fusion protein was assigned SEQ ID NO:2. The peptide between amino acids 1-49 of urokinase A chain (designated ATF) and L-methioninase is a flexible linker designed to join the two proteins without disturbing their function and is not susceptible to cleavage by host proteases (Argos et al., 1990). The rationale for the sequence of this fusion protein is as follows.

(1) Amino acids 1-49 of the urokinase A chain (denoted ATF; SEQ ID NO:3 for amino acid and SEQ ID NO:4 for nucleic acid) are used since this includes residues 12-32 (SEQ ID NO:11) that have been shown to be critical for binding to the urokinase receptor (Apella et al., 1987). The kringle domain of the urokinase A chain is excluded because this domain has been shown to bind heparin, which could bind polyanionic molecules such as the proteoglycans and aid in the invasion of tissue (Stephens et al., 1992). Adding on to the N-terminus of L-methioninase should give an active enzyme, since it was reported that an N-terminal addition to L-methioninase from T. vaginalis resulted in high enzyme activity toward methionine (McKie et al., 1998). Since the fusion proteins will be produced in recombinant Escherichia coli, the threonine at residue 18 of the uPA fragment will not be fucosylated; thus, the uPA fragment will not have the undesirable cell-proliferation property of the corresponding human uPA fragment (Rabbani et al., 1992). Bacteria, such as E. coli, do not carry out post-translational glycosylations, such as fucosylation.

(2) The peptide Gly-Ser-Gly-Ser-Gly (SEQ ID NO:9) has been determined by Argos (1990) as an optimal linker for joining proteins passively without disturbing their function and that is not susceptible to cleavage by host proteases. An additional Ser was added at the C-terminus of this peptide to create a BamHI restriction site in the gene (by selection of the codons for Gly-Ser). The Gly and Ser residues in this linker are the ones most preferred by natural linkers and impart some flexibility and yet maintain stability and conformation in solution through hydrogen bonding to water or the main chain. The amino acid sequence of the linker used in the fusion protein of the present invention has been assigned SEQ ID NO:5, and the nucleic acid sequence thereof has been assigned SEQ ID NO:6.

(3) ATF was placed at the N-terminus of the fusion protein since this is the same position that was successfully used for the binding peptide or protein for several fusion proteins containing Pseudomonas exotoxin (Pastan et al., 1992).

The amino acid sequence of the L-methioninase from Pseudomonas putida used in the fusion protein of the present invention has been assigned SEQ ID NO:7, while the nucleic acid sequence encoding such amino acid sequence has been assigned SEQ ID NO:8.

The construction of the fusion protein gene was carried out as follows: the ATF gene was amplified by PCR from the plasmid pULB1221 with a EcoRI restriction site added at the 5′ end and the flexible linker and a HindIII site added at the 3′ end. The L-methioninase gene contained in pKK223-3 was amplified by PCR with a BamHI site added at the 5′ end and a HindIII site at the 3′ end. PCR was performed using the Expand™ High Fidelity PCR system (Boehringer Mannheim, Indianapolis, Ind.). After digestion with the appropriate restriction enzymes, PCR gene fragments were agarose gel purified prior to ligation according to the Geneclean protocol (BIO101, Vista, Calif.). The digested and purified PCR fragments were directionally ligated into expression vector pKK223-3, which had been digested with EcoRI and HindIII and then purified by the Geneclean procedure. E. coli JM105 cells were transformed with the recombinant plasmid by electroporation.

A clone harboring the recombinant plasmid was grown to mid-log phase (OD_{600 nm}=0.5) at 37° C. in shake flasks in 250 ml of LB medium containing 100 μg ampicillin and 1% glucose. At this point, the culture was induced with isopropyl-β-D-thiogalactoside (IPTG) at 1 mM, and an additional 100 μg ampicillin was added. The cells were grown for an additional 5 h and then harvested by centrifugation. The pellet was resuspended in 10 ml of purification buffer at pH 8.0 (0.05 mM TPCK (N-p-tosyl-1-phenalanine chloromethyl ketone), 1 mM PMSF (phenylmethylsulfonyl fluoride), 1% ethanol, 1 mM EDTA (ethylenediamine tetraacetic acid), 0.02 mM pyridoxal phosphate, 0.01% β-mercaptoethanol, 0.02 M Tris, pH 8.0). The suspended cells were sonicated at 4° C. for a total time of 2.5 min at 4.5 W/ml (550 Sonic Dismembrator, Fisher Scientific, Pittsburgh, Pa.). The lysate obtained was centrifuged at 12,000×g for 30 min to remove the cell debris and then was subjected to a heat treatment by holding at 50° C. for 8 min and then cooling to 4° C. Subsequent steps were carried out at 4° C. The lysate was fed onto a 40 ml column (2.5 cm diameter) of Q Sepharose™ Fast Flow anion exchange adsorbent (Amersham Biotech, Piscataway, N.J.) equilibrated with the purification buffer at pH 8.0, and the column was eluted with a linear gradient of 0-0.8 M KCl in purification buffer over 2 h at a superficial velocity of 30 cm/h. Ammonium sulfate was added to give 35% saturation to the pool of the fractions containing the fusion protein, and the precipitate was removed by centrifugation at 10,000×g. The supernatant was fed onto a 30 ml column (2.5 cm diameter) of Phenyl Sepharose™ 6 Fast Flow (Amersham Biotech, Piscataway, N.J.) equilibrated with purification buffer at pH 6.5 and 35% saturated with ammonium sulfate. After washing the column with the same buffer that was 35% saturated with ammonium sulfate, the column was eluted with the same buffer with no ammonium sulfate. Both washing and elution for the hydrophobic interaction chromatography were at a superficial velocity of 30 cm/h. The fractions containing the fusion protein were dialyzed against purification buffer at pH 6.5 (0.05 mM TPCK, 1 mM PMSF, 1% ethanol, 1 mM EDTA, 0.02 mM pyridoxal phosphate, 0.01% β-mercaptoethanol, 0.02 M BisTris, pH 6.5). The dialyzed solution at pH 6.5 was fed onto the same anion exchange column as before, but with the column equilibrated with purification buffer at pH 6.5. The column was eluted at the same conditions as for the anion exchange chromatography at pH 8.0, except a 0-0.4 M KCl linear gradient was used. Fractions containing the fusion protein were pooled.

The enzymatic activity of L-methioninase was measured using L-methionine as a substrate and spectrophotometrically following the production of α-ketobutyrate with 3-methyl-2-benzothiazolone hydrazone hydrochloride (Esaki et al., 1973). Total protein was determined using the Bradford assay in a kit with bovine serum albumin as a standard (Bio-Rad, Richmond, Calif.). The Bradford protein assay was chosen because it gave much better protein balances around purification steps than the bicinchoninic acid (BCA) protein assay. Samples were analyzed by denaturing gel electrophoresis using the sodium dodecylsulfate-polyacrylamide gel electrophoresis (SDS-PAGE) method with staining by Coomassie blue (Laemmli et al., 1970). SigmaGel™ software (SPSS Science, Chicago, Ill.) was used to read band densities of Coomassie stained gels. Amino-terminal protein sequencing was performed by the Molecular Biology Resource Facility at the University of Oklahoma, Health Sciences Center on a protein sequencer equipped with an on-line PTH-amino acid analyzer (Procise model 492 sequencer, with model 610A data system, Perkin-Elmer, Applied Biosystems Division, Foster City, Calif.).

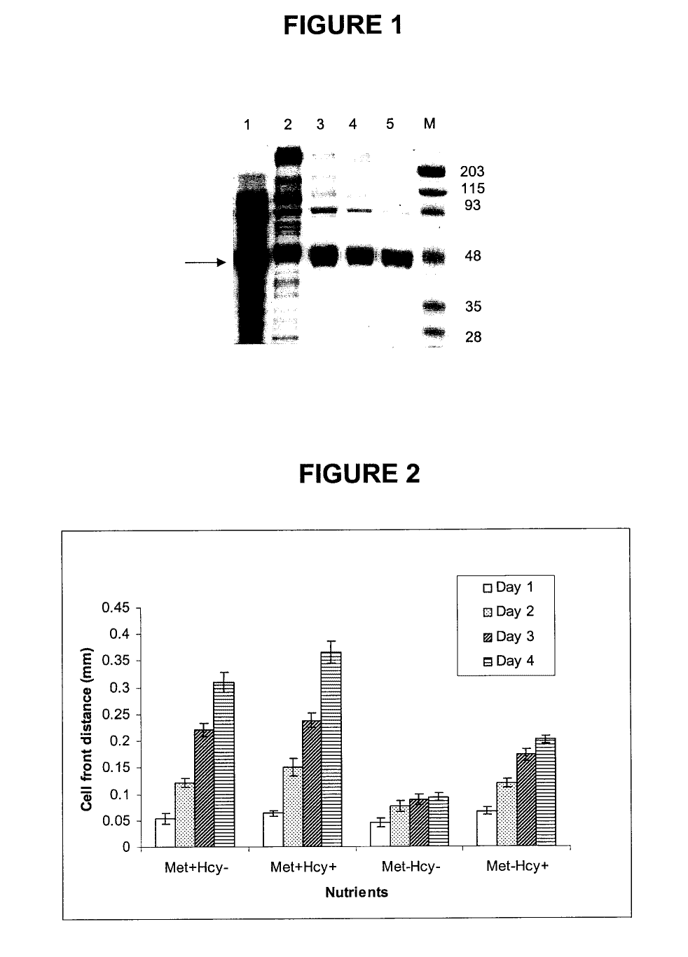

The SDS-PAGE results in FIG. 1 show the over-expression of the ATF-methioninase fusion protein in a clone containing the recombinant plasmid and the increasing purity of the fusion protein as the purification progresses. The purity of the fusion protein in the pooled fractions from the final chromatography was estimated to be 98% using the SigmaGel densitometry software, and the specific L-methioninase activity for these pooled fractions was 3.6 units/mg total protein (18 times higher than the specific L-methioninase activity in the starting cell lysate). The recovery of L-methioninase activity during purification was measured to be 29%.

Sequencing of the purified fusion protein was performed on the first eight amino-terminal amino acids. The sequencing results showed that the sequence was identical to the amino-terminus of the urokinase A chain (Ser-Asn-Glu-Leu-His-Gln-Val-Pro; SEQ ID NO:10) for 80±5% of the protein. One of the minor sequences obtained, for 10% of the protein, was the same as the sequence of the urokinase A chain starting at amino acid 21, which indicates that the first 20 amino acids were cleaved off (between Val and Ser) to give this sequence. This apparent cleavage of the fusion protein may explain the width of the band corresponding to the fusion protein on the SDS-PAGE analysis (FIG. 1). Thus, the sequences for the fusion protein, either whole or with the first 20 amino acids cleaved off, account for 90±5% of the protein, which is close to the purity determined by SDS-PAGE when error in the sequencing results is taken into account.

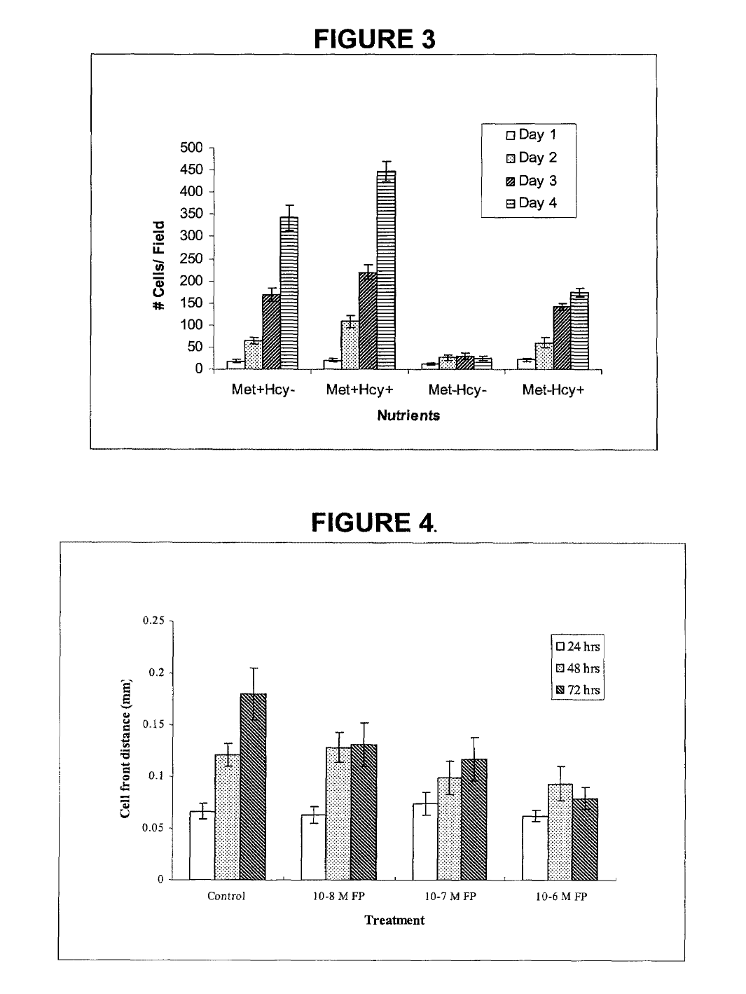

Methionine Dependency of MCF-7 Human Breast Cancer Cells. In order to determine the methionine dependency of the MCF-7 cell used in this study, the growth of cells in complete media and methionine free media was compared. The results shown in FIGS. 2 and 3 indicated that cell migration and proliferation index was significantly reduced in the absence of methionine at days 14 (p<0.05). In addition, the addition of homocystine improved cell migration and proliferation, but did not reverse the effects of a methionine deficiency.

Inhibitory Effects of ATF-methioninase—Cell Migration and Proliferation. Cell migration and proliferation index were evaluated using the culture wounding assay. In previous studies this method has been used to quantify growth factor-mediated stimulation of MCF-7 cell migration (Nguyen et al., 2002). Three days after seeding 5×10⁵ MCF-7 cells into 60 mm culture dishes, the cells were approximately 90% confluent. The cultures were wounded, washed three times with phosphate buffered saline (PBS), and treated with media containing either various concentrations of the fusion protein, methionine, or homocystine. In each experiment control cultures received RPMI media alone. At 24, 48 and, in some cases, 72 and 96 h following treatment, cell migration and proliferation index were determined by measuring both the distance traveled by the cell front into the wounded area (migration) and the number of cells in the wounded area (proliferation index)/microscopic field. Measurements were taken from 10-12 individual microscopic fields in each experiment, and data was summarized from 2-3 experiments.

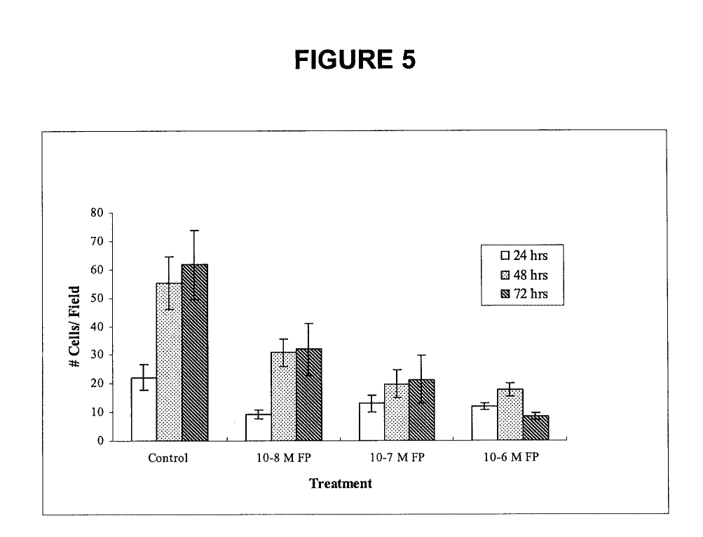

The effects of the fusion protein were examined over a concentration range of 10⁻⁶ to 10⁻⁸ M as shown in FIGS. 4 and 5. In these experiments, the fusion protein produced a dose-related inhibition of both the migration and proliferation index of MCF-7 cells on days 2 and 3 following fusion protein treatment (p<0.05). On day 1, the fusion protein-induced inhibition of cell migration and proliferation index was not found to be dose-related.

Cell Binding Assay—Specific Binding of ATF-methioninase to MCF-7 Cells. The relative binding of the fusion protein to urokinase receptors was carried out by measuring the displacement of fusion protein. Fusion protein displacement was quantified by measuring L-methioninase activity of the supernatant solution in response to increasing concentration of pure urokinase. MCF-7 cells were plated in 96 well plates containing 10³ cells/well, a saturating concentration of fusion protein (10⁻⁶ M), and pure urokinase over a range of 3×10⁻¹⁰ to 3×10⁻⁶ M. Following a one hour incubation at 37° C. in a CO₂ incubator, the supernatant was removed and centrifuged to remove all cellular debris. The supernatant L-methioninase concentration was measured using a spectrophotometric method as previously described (Esaki et al., 1973). The urokinase concentration which produced a 50% displacement of fusion protein in this assay was used to estimate the relative binding affinity as previously described (Jain et al., 1997).

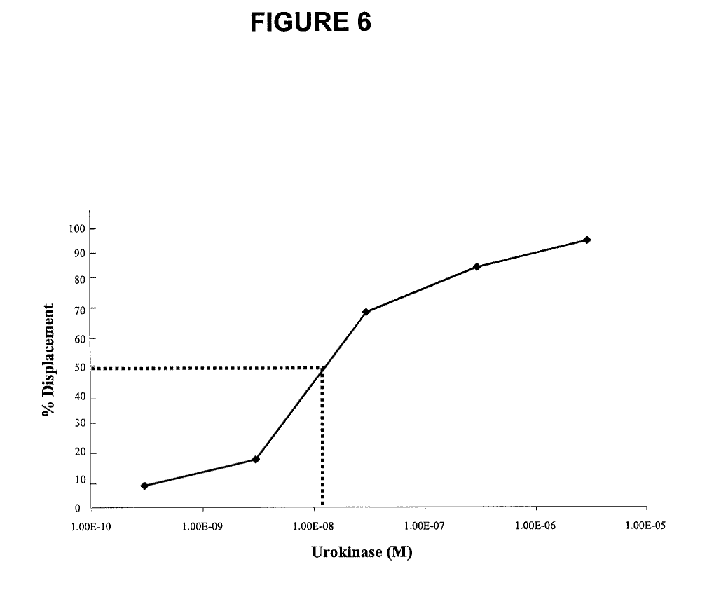

In these experiments, the displacement of the fusion protein from urokinase receptors on MCF-7 cells was determined. As shown in FIG. 6, increasing concentrations of urokinase over a concentration range of 3×10⁻¹⁰ to 3×10⁻⁶ M produced a dose-related displacement of fusion protein. The urokinase concentration necessary to produce a 50% displacement of fusion protein was determined to be approximately 10⁻⁸ M. It was also determined that human EGF, over the same concentration range, did not produce any significant displacement of the fusion protein.

Growth and Metastasis of Breast Tumor Cells in Nude Mice. The nude mouse xenograft model was used to determine the influence of the fusion protein upon the growth and metastasis of human breast tumor cells in vivo. MCF-7 human breast cells (10⁶ cells), suspended in Matrigel were injected into the flank of nude mice. These cells were stably transfected with the β-galactosidase (β-gal) reporter gene so that tumor metastasis could be determined and quantified. The development of tumor masses was monitored over a period of 30 days. The animals were then randomly placed into treatment groups. Treatment groups received either the fusion protein (three mice each treated with 12 μl/day at 5×10⁻⁶ M, equal to 12 μg/day assuming a molecular weight of 196,000 Da for the homotetrameric fusion protein) or vehicle in the control group (two mice) administered by continuous infusion over a period of 14 days using an Alzet osmotic infusion pump. (This dosage level was selected because it is approximately equivalent to the concentration of 10⁻⁸ M used in the in vitro studies, which was the lowest concentration that showed inhibition of cell proliferation—see FIG. 5.) The pump was implanted subcutaneously and delivered the fusion protein or vehicle directly to the tumor site. The animal weights were recorded twice weekly, and tumors were measured by caliper and tumor volumes calculated using the formula: volume=length×width²×0.4. At the conclusion of the 14-day infusion period, the animals were anesthetized and killed by cervical dislocation. Tumor and lung tissue were excised and weighed, and all animals were examined for organ and tissue cytotoxicity. The β-gal activity of the tissue samples was measured to quantify tumor growth and metastatic development.

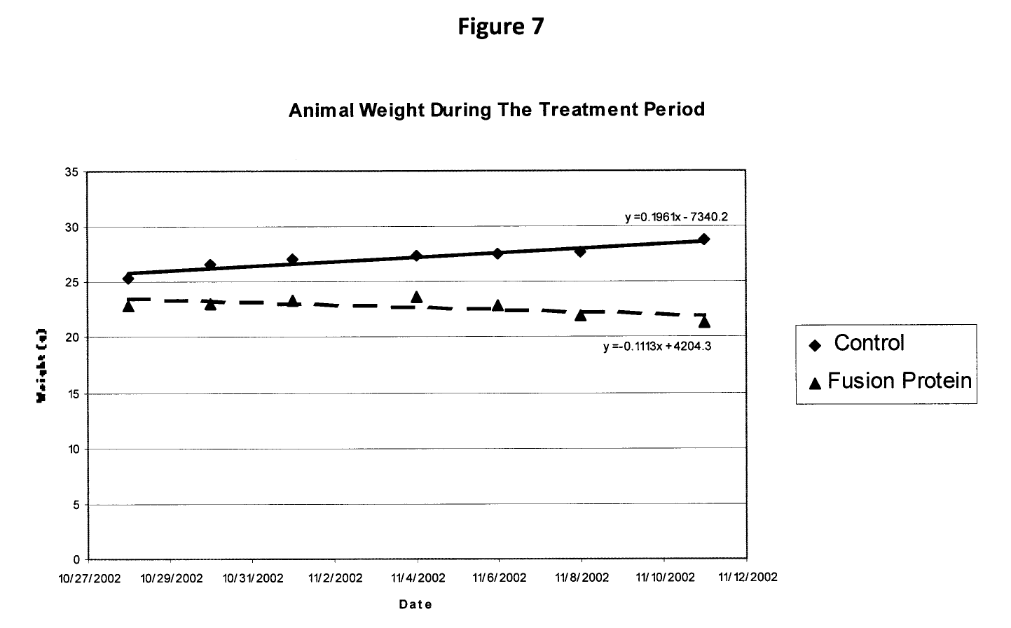

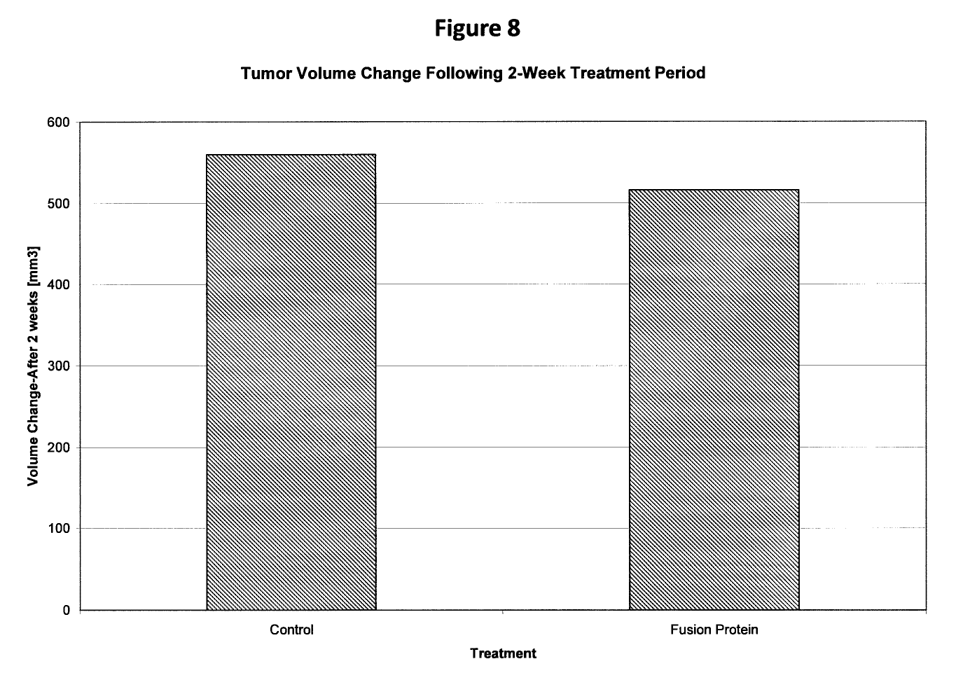

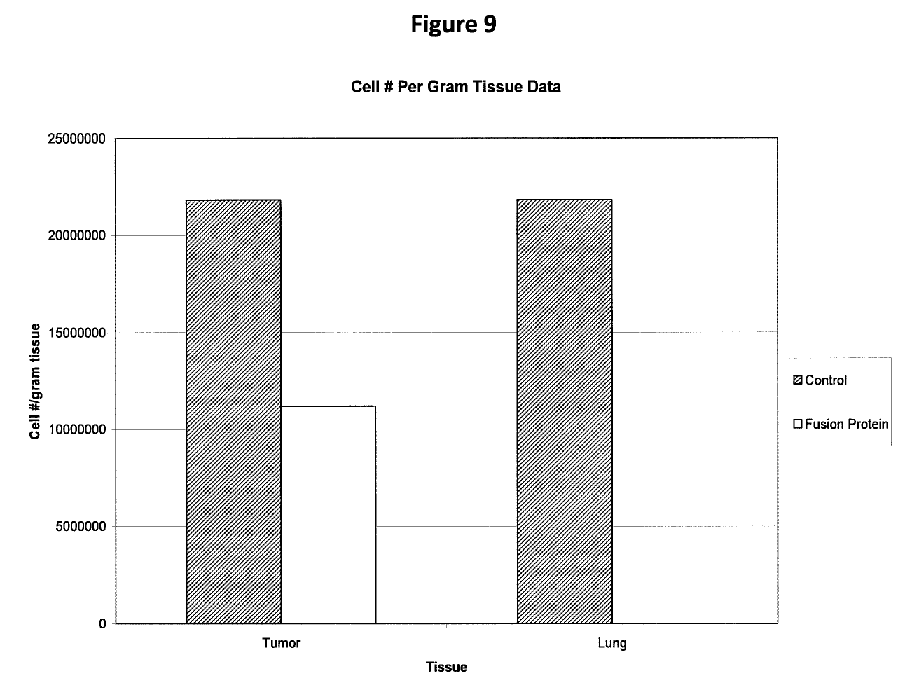

The results demonstrate that the fusion protein was not cytotoxic to the nude mice since none of the treated animals died or showed signs of whole animal or organ cytotoxicity during the 14-day treatment period. The fusion protein treated animal weight remained unchanged while the control animals gained 2-3 grams (FIG. 7). The increase in tumor mass in the control and treated animals was approximately the same over the 14-day treatment period (FIG. 8). However, the total number of cancer cells/gram of tissue was significantly reduced in the fusion protein treatment group (FIG. 9). Further, lung metastases were found in all of the control animals, while none were found in the fusion protein treated mice (FIG. 9).

The dosage level of 12 μg/day corresponds to 0.53 mg/kg/day based on the average animal weight, or a cumulative dosage of 7.4 mg/kg for the entire period of treatment. By comparison, the dosage level of L-methioninase used in the study of Kokkinakis et al. (1997b) to treat mice with implanted human medulloblastoma in combination with dietary restrictions of methionine, homocystine, and choline was much higher, 44 mg/kg/day. In addition, the dosage level of the bacterial enzyme L-asparaginase in the treatment of humans with acute lymphocytic leukemia is 1.8 mg/kg/day for 10 days (Ylikangas et al., 2000; Drug Information, 2003), or a cumulative dosage of 18 mg/kg. Thus, the cumulative dose of the fusion protein, based upon the weight of the subject that has been found to have an effect, is low compared to the standard dose for L-asparaginase.

The above described Example provides an ATF-methioninase fusion protein constructed by ligating the gene for the first 49 amino acids of the urokinase A chain to a gene for L-methioninase from Pseudomonas putida, with the gene coding for a six amino acid flexible linker in between. This fusion protein, which had L-methioninase activity, was produced in E. coli in soluble form and purified to near homogeneity with three chromatography steps.

The MCF-7 human breast cancer cells used in the biological testing were verified to be methionine dependent, as demonstrated by the reduction in cell migration and proliferation index when the amino acid methionine is replaced by homocystine (FIGS. 2 and 3). Normal human cell lines survive and grow well with this substitution. The ATF-methioninase fusion protein inhibited the migration and proliferation index of MCF-7 cells over a concentration range of 10⁻⁶ to 10⁻⁸ M in a dose-dependent manner over a period of 3 days (FIGS. 4 and 5). To show that ATF-methioninase would bind specifically to MCF-7 cells, a binding assay was performed by saturating the cells with the fusion protein and adding urokinase at various concentrations. The relative affinity of ATF-methioninase for the cells (50% competition at 10⁻⁸ M, FIG. 6) is 10-fold lower than that reported by others for the displacement of a larger ATF (1-135) by urokinase from cultured cells (Stoppelli et al., 1985), which is consistent with reduced binding strength for other urokinase ATF's that are less than 135 amino acids in size (Appella et al., 1987).

In a study of the effect of the fusion protein in nude mice with MCF-7 human breast tumors, the findings were as follows: (1) the fusion protein infusion was not acutely cytotoxic to the nude mice, (2) the fusion protein did not reduce the growth of an established breast tumor mass (growth was slightly smaller but not considered significant), (3) the fusion protein significantly reduced the concentration of tumor cells within the primary tumor mass, and (4) the fusion protein prevented the development of lung metastasis in this animal model. Findings (1), (3), and (4) are very encouraging, since one of the primary goals of the present invention is to prevent cancer metastasis without being toxic to normal cells. Finding (3), coupled with in vitro cell proliferation studies (see FIG. 5), suggests that the dosage needs to be higher in the studies with mice in order to achieve significant reduction in tumor growth.

The major mechanism of the inhibitory effects of ATF-methioninase of the present invention is believed to be the methioninase-induced depletion of methionine available to the cells. Another possible mechanism of ATF-methioninase inhibition of cell migration and proliferation may be related to the specific binding to, and inactivation of, the urokinase receptor. Since urokinase is known to be involved in cancer cell invasion, specific binding to this receptor, by the fusion protein, may inhibit or alter urokinase related activity.

Urokinase or ATF have been fused to the cytotoxic proteins saporin (Cavallaro et al., 1993) and diphtheria toxin (Vallera et al., 2002). While these fusion proteins were found to be cytotoxic to cancer cells, they would also kill normal cells that also have urokinase receptors, such as neutrophils, eosinophils, monocytes, and fibroblasts. The ATF-methioininase fusion protein is advantageous in this respect, since the growth of normal cells would not be inhibited.

None of the work to date with L-methioninase treatment of cancer cells has involved the targeting of the L-methioninase to the cell surface. Advantages that are foreseen for targeting L-methioninase to the cell surface include the following: Since the L-methioninase is being targeted to the cells that it is intended to kill, the dosages of this fusion protein containing L-methioninase should be much lower than when L-methioninase alone is administered systemically. In addition, it may be possible to avoid having dietary restrictions of methionine, homocystine, and choline and injections of homocystine as were needed in the previous studies with mice (Kokkinakis et al., 1997b).

In conclusion, the results of this Example described herein above demonstrate that an ATF-methioninase fusion protein is capable of inhibiting both the proliferation and migration of human breast cancer cells. In addition, the Example indicates that the fusion protein is specifically targeted to the urokinase receptor of the cancer cells. This fusion protein can serve as a prototype for targeting methioninase and/or other anticancer agents to cancer cells.

### Example 2

The work described in Example 1 demonstrates that ATF-methioninase fusion protein specifically binds to the urokinase receptor on MCF-7 breast cancer cells in vitro, based on the measurement of ATF-methioninase displaced by urokinase at various concentrations from the surface of the MCF-7 cells. ATF-methioninase produced a dose-dependent inhibition of both the proliferation and migration of MCF-7 cells in vitro over a period of 1 to 3 days.

Because of these results with ATF-methioninase, an expanded study was performed that compares the effect of ATF-methioninase on MCF-7 in vitro cell proliferation and migration to L-methioninase without the receptor targeting peptide added, and to ATF-methioninase with the methioninase mutated so that there is no enzymatic activity. The effect of ATF-methioninase on the cell proliferation and migration of PC-3 prostate cancer and SK-LU-1 lung cancer cells was also studied. To compare the effect of ATF-methioninase with a fusion protein that is internalized, a TGF-methioninase fusion protein (transforming growth factor-linked to L-methioninase) was produced and tested on MCF-7 breast cancer cells in vitro.

To provide more evidence of binding to the cancer cell surface, immunocytochemical localization using an anti-L-methioninase antibody was utilized to observe the binding of ATF-methioninase to MCF-7 cells in vitro. An in vivo study of the effect of intra-tumoral injection with either ATF-methioninase or L-methioninase on the growth of MCF-7 tumor xenografts in nude mice was also performed.

### Materials and Methods

Materials. Synthetic oligonucleotides were produced by the Molecular Biology Resource Facility at the University of Oklahoma Health Sciences Center. Vector pET-30 EK/LIC, HRV 3C protease, and NovaBlue and BL21(DE3) E. coli cells were obtained from Novagen (Madison, Wis.). The gene for transforming growth factor-α (TGF) on plasmid pVC 387 was obtained from Dr. Ira Pastan at the National Cancer Institute.

Construction of recombinant expression plasmids. The expression vectors pET-30/Ek/LIC/ATF-Meth and pET-30/Ek/LIC/Meth were constructed as follows: The DNA sequences encoding the ATF-methioninase fusion protein and L-methioninase were amplified from pKK223-3/ATF-Meth (Peron et al., 2003) by the polymerase chain reaction using the Expand High Fidelity PCR system (Boehringer Mannheim, Indianapolis, Ind.). The sequence of ATF-methioninase is as follows: N-(amino acids 1-49 of human urokinase A chain)-Gly-Ser-Gly-Ser-Gly-Ser-(L-methioninase from P. putida). The peptide between ATF and L-methioninase is a flexible linker designed to join the two proteins without disturbing their function and is not susceptible to cleavage by host proteases (Argos, 1990). Forward and reverse primers used for PCR for ATF-methioninase and L-methioninase were as follows:
(1) the primer ATF 5′ (GACGACGACAAGATGCTTGAAGTCCTCTTTCAGGG ACCCAGCAATGAACTTCATCAAGTTCC) (SEQ ID NO:12) introduced at the 5′ end of the ATF-methioninase DNA sequence an LIC cloning site (italics) and an HRV 3C protease site (underlined);
(2) the primer METH 3′ (GAGGAGAAGCCCGGTTATCATGCACACGCCTCC AATGCCAACTCG) (SEQ ID NO:13) introduced at the 3′ end of the ATF-methioninase or L-methioninase DNA sequence an LIC cloning site (italics);
(3) the primer METH 5′ (GACGACGACAAGATGCTTGAAGTCCTCTTTCA GGGACCCCGCGACTCCCATAACMCACC) (SEQ ID NO:14) introduced at the 5′ end of the L-methioninase DNA sequence an LIC cloning site (italics) and an HRV 3C protease site (underlined).

PCR gene fragments were agarose gel-purified prior to ligation according to the Qiagen protocol (BIO101, Vista, Calif.). The PCR product was annealed to the pET-30 EK/LIC linear vector and transformed into NovaBlue cells according to the Novagen protocol. This construction results in an N-terminal His-tag sequence with an integrated thrombin cleavage site, enterokinase cleavage site, and an engineered HRV 3C protease cleavage site next to the start of ATF or L-methioninase. (HRV 3C protease cleaves the sequence LEVLFQ↓GP). DNA sequences were verified by sequencing at the Oklahoma Medical Research Foundation (Oklahoma City).

The gene for the fusion protein N-(TGF-α)-Gly-Ser-Gly-Ser-Gly-Ser-(L-methioninase)-C (SEQ ID NO:15, amino acid sequence thereof; SEQ ID NO:16, nucleotide sequence thereof) was inserted into the expression vector pET44/Ek/LIC/TGF-Meth as follows: The DNA sequence encoding the L-methioninase was amplified by polymerase chain reaction (PCR) from pKK223-3/ATF-Meth with a BamHI site at the 5 end and a LIC site at the 3 end, and the DNA sequence encoding TGF-α was amplified by PCR from pVC 387 with the LIC site and HRV 3C protease site at the 5′ end and the flexible linker and BamHI site at the 3′ end. PCR was performed using the Expand High Fidelity PCR system (Boehringer Mannheim, Indianapolis, Ind.). Forward and reverse primers used for PCR for TGF-methioninase were as follows:
(1) the primer TGF 5′ (GACGACGACAAGATGCTTGAAGTCCTCTTTCAGGG ACCCGGAGTGGTGTCCCATTTTAATGACTGCCC) (SEQ ID NO:17) introduced at the 5′ end of the TGF DNA sequence an LIC cloning site (italics) and an HRV 3C protease site (underlined);
(2) the primer TGF 3′(GCGGATCCAGAACCGCTGCCAGCCAGGAGGTCC GCATGCTCACAGCG) (SEQ ID NO:18) introduced at the 3′ end of the TGF DNA sequence an BamHI site (bold and italics) and flexible linker region (underlined);
(3) the primer METH 5′ (CGCGGATCCCGCGACTCCCATAACAACACC) (SEQ ID NO:19) introduced at the 5′ end of the L-methioninase DNA sequence an BamHI site (bold and italics).
(4) the primer METH 3′(GAGGAGAAGCCCGGTTATCATGCACACGCCT CCAATGCCAACTCG) (SEQ ID NO:20) introduced at 3′ end of the L-methioninase DNA sequence an LIC cloning site (italics).

PCR gene fragments were agarose gel-purified prior to restriction enzyme digestion according to the Qiagen protocol (BIl0101, Vista, Calif.). After digestion of these gene fragments with BamHI enzyme, these were purified using PCR purification kit (Qiagen, BIO101, Vista, Calif.). The two fragments were ligated using T4 DNA ligase. Ligated product was purified according to the Qiagen protocol (BIO101, Vista, Calif.). The ligated product annealed to the pET-44 EK/LIC linear vector and transformed into NovaBlue cells according to the Novagen protocol. This construction results in N-terminal His-tag and NusA-tag sequences with an integrated thrombin cleavage site, enterokinase cleavage site, and an engineered HRV 3C protease cleavage site next to the start of TGF-methioninase. DNA sequences were verified by sequencing at the Oklahoma Medical Research Foundation (Oklahoma City).

Site-directed mutagenesis of ATF-methioninase. The plasmid pET30 Ek/LIC/ATF-Meth was used as a template for site-directed mutagenesis using PCR. The forward and reverse primers that are complementary to each other and create a mutation at the 114th residue of L-methioninase (Y114F) are as follows:

(a) the primer mMETH
5′ (GCGCACCTTGTTTGGCTGCACCTTTG (SEQ ID NO: 21));
(b) the primer mMETH
3′ (CAAAGGTGCAGCCAAACAAGGTGCGC (SEQ ID NO: 22)).

Tyr114 is in the active site of L-methioninase, and the Y114F mutation has been shown to greatly reduce enzymatic activity toward methionine (Inoue et al., 2000). PCR was performed according to Stratagene protocol (La Jolla, Calif.). Colonies with the mutation were identified by DNA sequencing, and plasmid from these colonies was purified and then transformed into BL21(DE3) cells.

Expression and purification of recombinant proteins. E. coli host strain BL21 (DE3) harboring ATF-methioninase, mutated ATF-methioninase, TGF-methioninase, or L-methioninase was grown in 50 ml of LB medium containing 35 μg/ml kanamycin (pET-30 vectors with the ATF-methioninase, mutated ATF-methioninase, or L-methioninase genes) or 50 μg/ml ampicillin (pET44 vector with the TGF-methioninase gene) overnight at 37° C. with shaking. This cell culture was added to 1 liter of fresh culture medium, and the culture was grown with shaking at 37° C. When the absorbance at 600 nm reached 0.5, recombinant protein expression was induced by adding isopropyl β-D-thiogalactopyranoside (IPTG) to 0.4 mM concentration, and shaking was continued at 30° C. for 5 hours, except for TGF-methioninase, where shaking was continued at 37° C. for 3 hours. The cell pellet was collected by centrifugation and was resuspended in 40 ml of sonication buffer (0.05 mM N-p-tosyl-L-phenylalanine chloromethyl ketone (TPCK), 1 mM phenylmethylsulfonyl fluoride (PMSF), 1% ethanol, 0.02 mM pyridoxal phosphate, 0.01% β-mercaptoethanol, 0.02 M sodium phosphate, pH 7.4). The suspended cells were sonicated at 4° C. for a total of 2.5 min at 4.5 W/ml (550 Sonic Dismembrator, Fisher Scientific, Pittsburgh, Pa.). The lysate obtained was centrifuged at 12,000 g for 30 min to remove the cell debris. Subsequent steps were carried out at 4° C.

After adding imidazole (40 mM) and NaCl (500 mM) to the lysate, it was fed to a 5 ml HisTrap chromatography column (Amersham Biotech, Piscataway, N.J.) equilibrated with wash buffer (20 mM sodium phosphate, 40 mM imidazole, 500 mM NaCl, 0.02 mM pyridoxal phosphate, pH 7.4). The column was washed with the wash buffer, and then His-tagged recombinant protein was eluted by elution buffer (20 mM sodium phosphate, 500 mM imidazole, 500 mM NaCl, 0.02 mM pyridoxal phosphate, pH 7.4). Eluted protein was dialyzed against 20 mM sodium phosphate buffer at pH 7.4 containing 0.02 mM pyridoxal phosphate. The cleavage of N-terminal His-tag was achieved by use of HRV 3C protease (Novagen, 0.5 U/mg protein substrate) with the recommended buffer added (1.5 M NaCl, 0.5 M Tris-HCl, pH 7.5) for 8 hours at 4° C. Cleaved protein was fed again onto a 5 ml HisTrap column, and pure protein was eluted in a linear gradient of 0-0.5 M imidazole. Purified protein was dialyzed against 20 mM sodium phosphate buffer at pH 7.4 containing 0.02 mM pyridoxal phosphate and 0.1 M NaCl, and this formulation was flash frozen using liquid nitrogen and then lyophilized in tubes at a concentration of 1-2 mg/ml.

The enzymatic activity of L-methioninase was measured using L-methionine as a substrate by the spectrophotometric determination of α-ketobutyrate with 3-methyl-2-benzothiazolone hydrazone hydrochloride (Esaki et al., 1987). Total protein was determined using the Bradford assay with bovine serum albumin as a standard (Bio-Rad, Hercules, Calif.). Samples were analyzed by denaturing gel electrophoresis using the SDS-PAGE method with staining by Coomassie blue (Laemmli, 1970). Amino-terminal protein sequencing was performed by the Molecular Biology Resource Facility at the University of Oklahoma Health Sciences Center.

Cell culture. MCF-7 and PC-3 human cancer cells were maintained as monolayer cultures in RPMI 1640 media (without phenol red) supplemented with 2 mM L-glutamine, gentamicin (50 μg/ml), penicillin (100 units/ml), streptomycin (100 μg/ml), estradiol (10⁻¹¹ M) (all from Sigma, St. Louis, Mo.), and 5% bovine calf serum (Hyclone, Logan, Utah). The media was filter sterilized and stored at 4° C. prior to use. It was previously determined that the cells used in this study are methionine dependent (Peron et al., 2003).

Cell Binding Assay. The specific binding of fusion proteins was measured by immunocytochemical localization. The immunocytochemistry was performed using a monoclonal antibody to L-methioninase, which has been produced in purified form for us (Sigma Genosys, St. Louis). The antibody was used in the immunocytochemical localization of each methioninase-containing fusion protein and L-methionase to the tumor cell surface. Since both fusion proteins to be examined in this study contain L-methioninase, this antibody and procedure were used for both fusions. This assay was performed on cells grown in culture and on cells grown in mouse xenografts. For the cell culture experiments, the cells were seeded in 35 mm culture dishes. In 2 days when the cells were approximately 60% confluent, cells were removed and placed on glass slides and prepared for immunocytochemistry as previously described using a methioninase-specific antibody (Zang et al., 2003). The slides were counterstained with hematoxylin (Vector Laboratories, Burlingame, Calif.), cleared with xylene, and coverslipped with Acrymount (StatLab, Lewisville, Tex.). Slides processed in the same manner, except without primary antibody, were included to exclude negative immuno-reactivity in these experiments.

Culture Wounding Assay. Cell migration and proliferation was evaluated using the culture wounding assay as previously described (Peron et al., 2003; and Zang et al., 2003). Three days after seeding 5×10⁵ cells into 60 mm culture dishes, the cells were approximately 90% confluent. The cultures were wounded, washed three times with PBS, and treated with media containing either various concentrations of the fusion proteins or L-methioninase. In each experiment control cultures received RPMI media alone. At 24, 48 and 72 hours following treatment, cell migration and proliferation were determined by measuring both the distance traveled by the cell front into the wounded area (migration) and the number of cells in the wounded area (proliferation)/microscopic field. Measurements were taken from 10-12 individual microscopic fields in each experiment, and data was summarized from 2-3 experiments.

Mouse Xenograft Assay. A mouse xenograft model was used to examine the effects of fusion protein treatment on MCF-7 tumor xenografts as previously reported (Buller et al., 2003). The cancer cells (5×10⁶), stably transfected with a β-gal reporter, were suspended in Matrigel and injected into the flank of nude mice. The development of tumor masses was monitored over a period of 25 days and the animals were randomly placed into treatment groups containing 10 animals/group. Treatment groups received 25 μl of 5×10⁻⁶ M fusion protein or L-methioninase solution or vehicle in the control group by intra-tumoral injection on alternate days. The animals were treated for a period of 20 days. At the beginning of the treatment period and at 3-day intervals, animal weights were recorded. At the end of the treatment period, the animals were killed. Tumor tissue was removed, and the cancer cells were quantified by colorimetric measurement the β-gal reporter in each sample. The animals were examined for organ and tissue cytotoxicity.

Data Analysis. Multiple group comparisons were conducted using ANOVA and Student's t-test for pair-wise comparisons. Group differences resulting in p values of less than 0.05 were considered to be statistically significant.

### Results

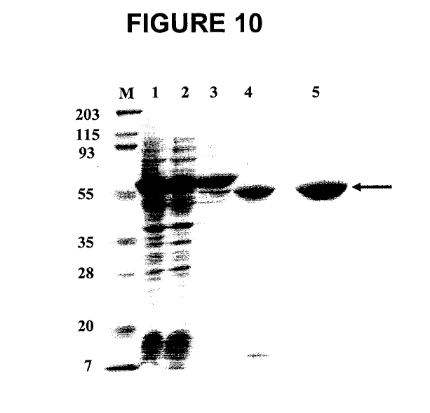

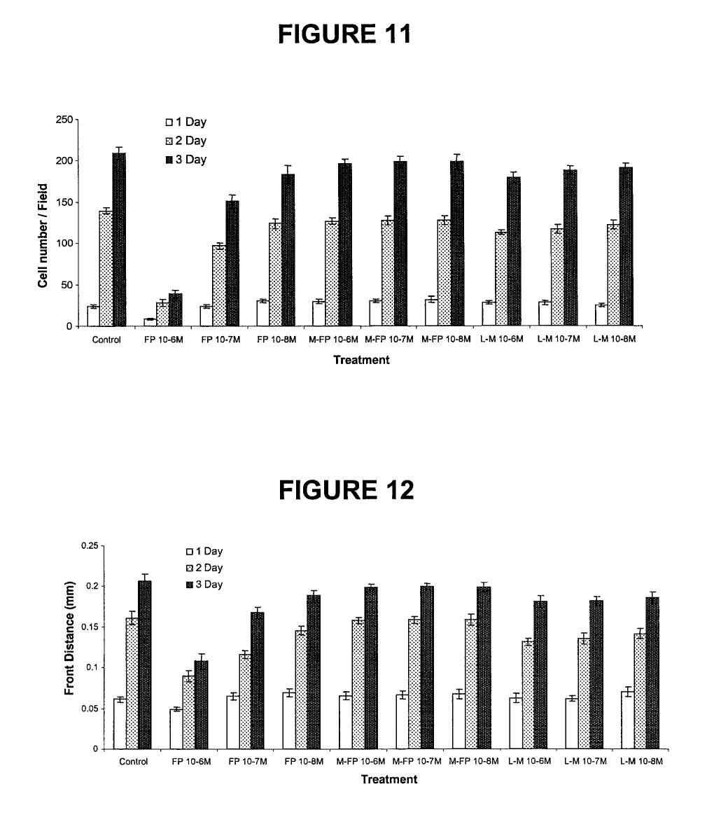

Expression and purification of ATF-methioninase, mutated ATF-methioninase, TGF-methioninase, and L-methioninase. All the proteins were expressed in soluble form 30° C. in E. coli BL21(DE3) cells after transformation with the recombinant plasmid (except for TGF-methioninase, which was expressed in soluble form at 37° C.; all other proteins were found to be insoluble when they were expressed at 37° C.). The SDS-PAGE results in FIG. 10 show the expression at 30° C. of the ATF-methioninase fusion protein in a clone containing the recombinant plasmid; the increasing purity of the fusion protein as the purification progressed is also shown. Complete cleavage at the HRV 3C protease site to remove the His-tag was obtained using HRV 3C protease (FIG. 10, lanes 4 and 5). The purity of the fusion protein from the final chromatography was estimated to be 94% using Quantity One densitometry software analysis of lane 5 of the SDS-PAGE gel in FIG. 10 (Bio-Rad, Hercules, Calif.). Similar expression and purity was also obtained with mutated ATF-methioninase, TGF-methioninase, and L-methioninase (results not shown). Amino acid sequencing of the purified fusion proteins was performed on the first eight amino-terminal amino acids. The sequence was identical to the amino-terminus of the urokinase A chain (Ser-Asn-Glu-Leu-His-Gln-Val-Pro (SEQ ID NO:10)) or TGF-α for 90±5% of the protein, which is consistent with the purity estimation of the SDS-PAGE gels of the purified proteins.

Specific L-methioninase activities of the purified proteins were as follows: 13.9 U/mg total protein for ATF-methioninase, 0 U/mg total protein for mutated ATF-methioninase, 12.5 U/mg total protein for TGF-methioninase, and 14.6 U/mg total protein for L-methioninase.

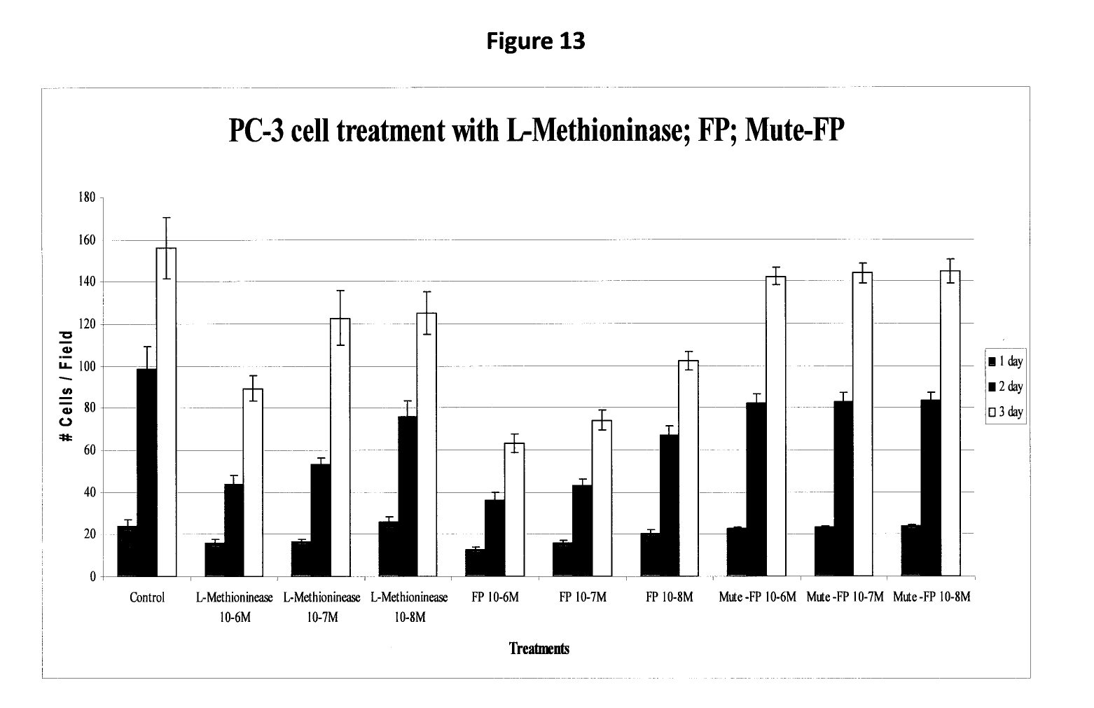

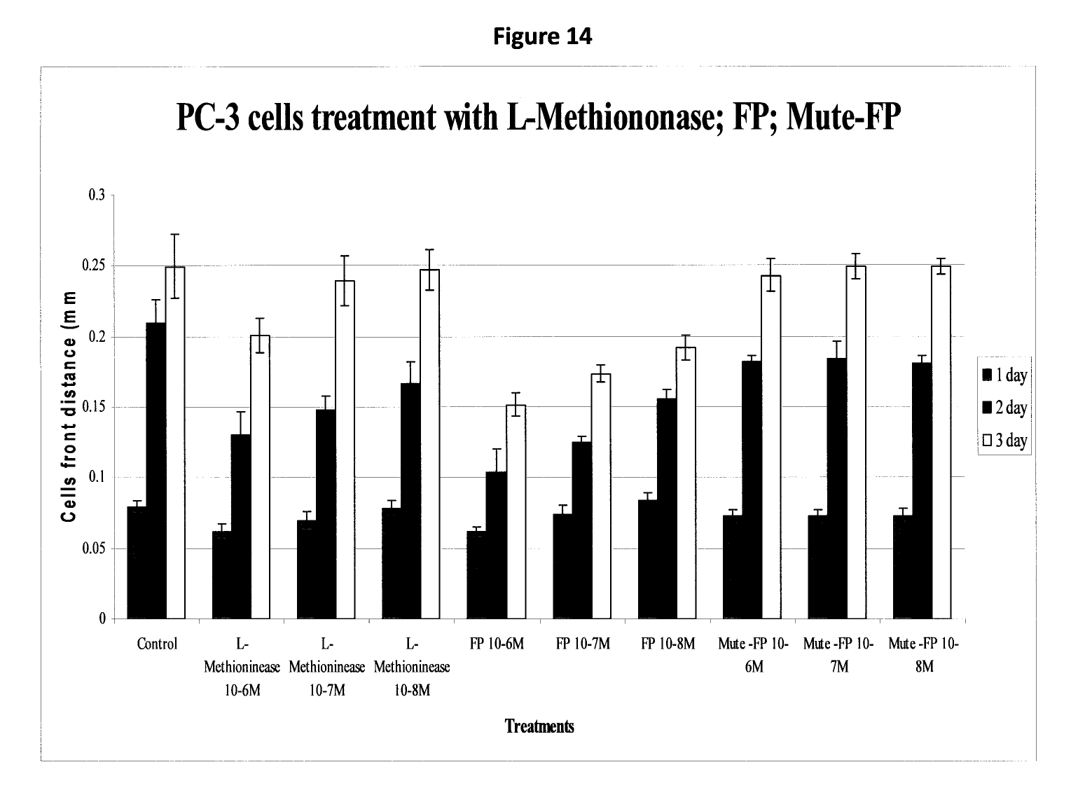

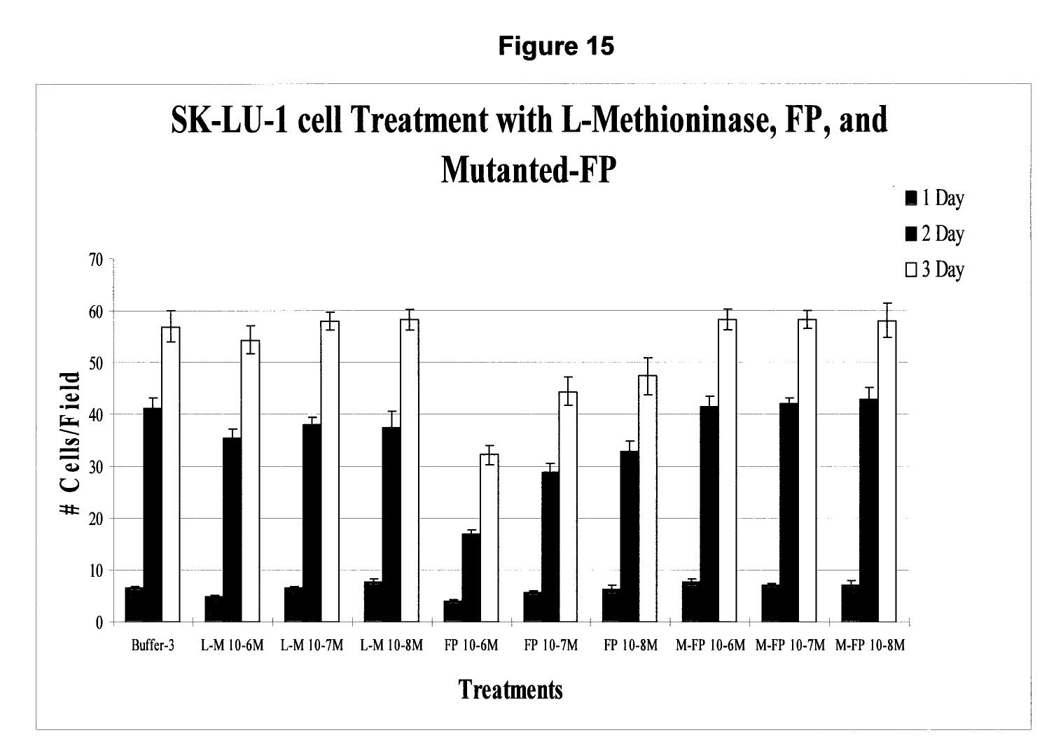

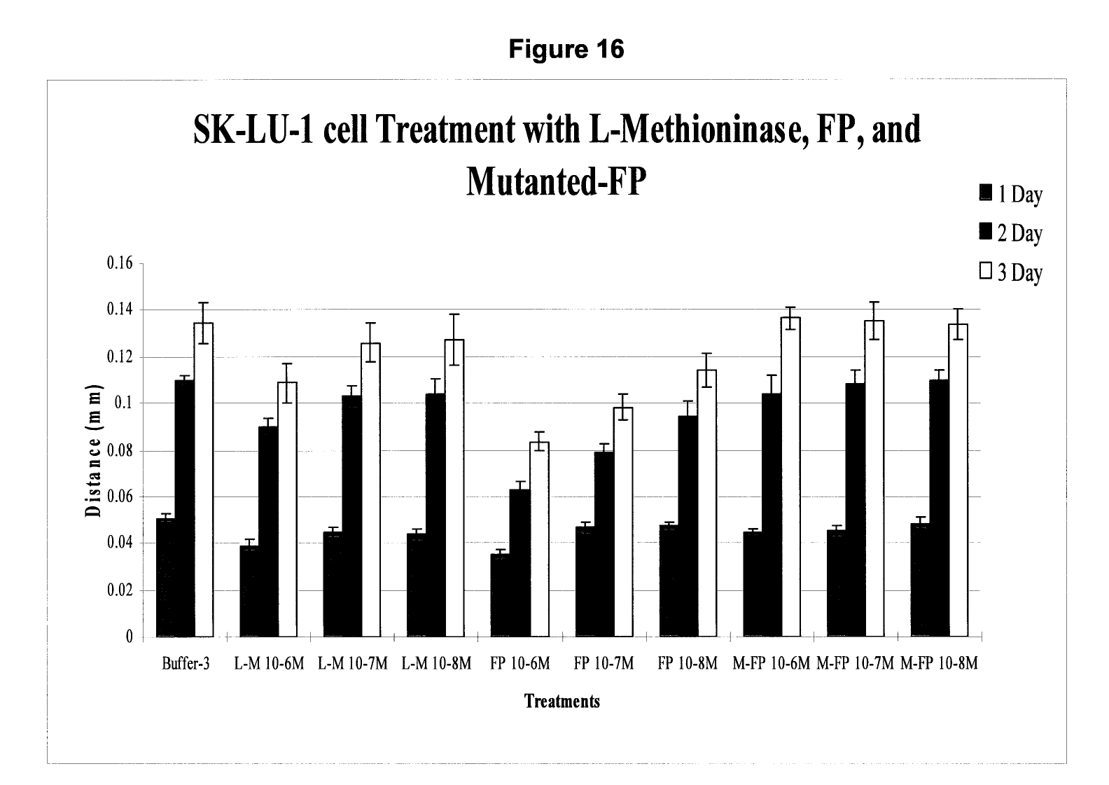

Inhibitory Effects of ATF-methioninase and TGF-methioninase fusion proteins, mutated ATF-methioninase, and methioninase. The effects of ATF-methioninase fusion protein were examined over a concentration range of 10⁻⁶ to 10⁻⁸ M as shown in FIGS. 11-16. In these experiments, the ATF-methioninase fusion protein consistently produced a dose-related inhibition of both the migration and proliferation of MCF-7, PC-3, and SK-LU-1 cells on days 2 and 3 following fusion protein treatment at all concentrations (p>0.05). The fusion protein with a mutated L-methioninase produced little or no inhibition of cell migration or proliferation over the same concentration range. Treatment of SK-LU-1 cancer cells with L-methioninase alone produced a significant inhibition (p<0.05) of cell migration on days 2 and 3 following treatment only at the highest concentration of 10⁻⁶ M, and proliferation was significantly inhibited (p<0.05) on day 2 only at 10⁻⁶ M but was not inhibited on day 3 at any of the concentrations. L-methioninase induced inhibition was much smaller than that produced by the fusion protein. Treatment of the PC-3 cancer cells with L-methioninase alone consistently produced a significant reduction in proliferation and migration on all three days only at the highest concentration of 10⁻⁶ M (p<0.05). The L-methioninase-induced inhibition was significantly less than that produced by the fusion protein on day 3 at all concentrations tested (p<0.05).

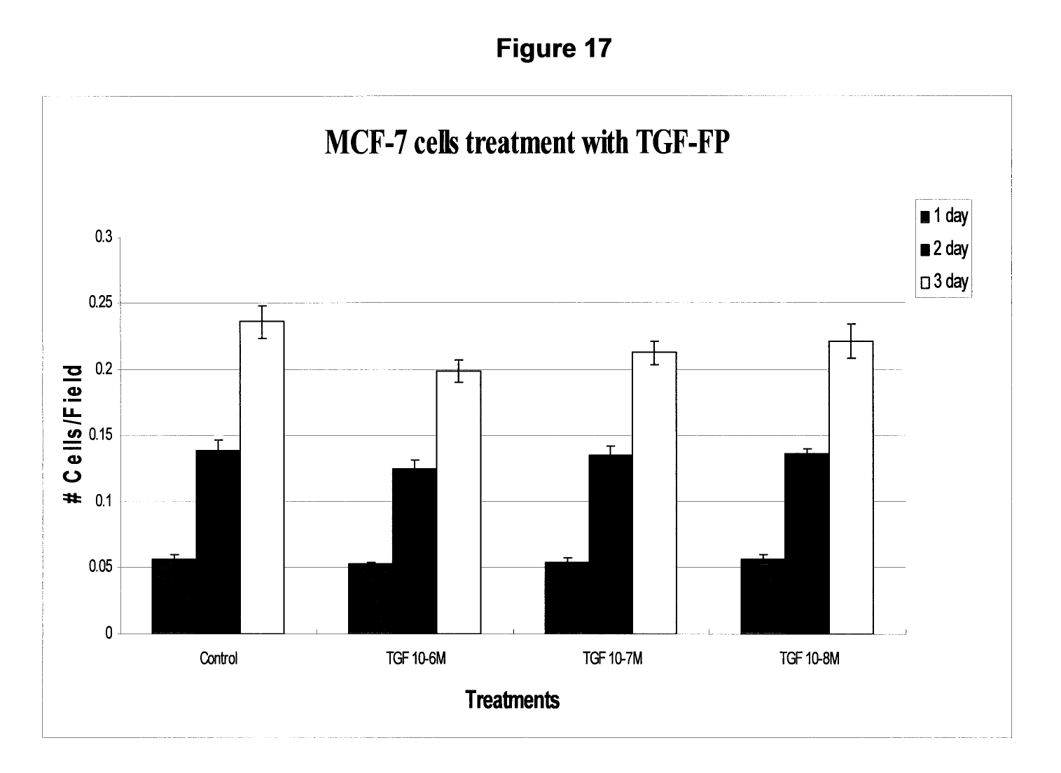

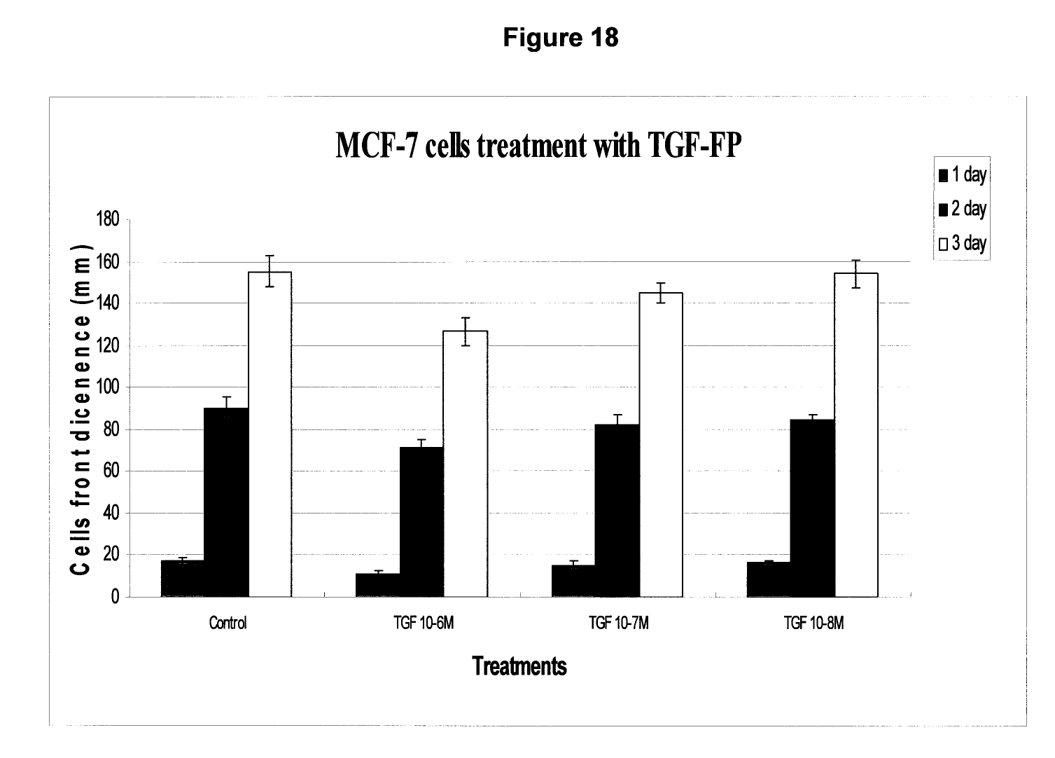

Next, it was determined whether L-methioninase is as effective as an anticancer agent when it is targeting to an internalizing receptor compared to when it is targeted to a non-internalizing receptor. The effects of the TGF-methioninase fusion protein on MCF-7 cells were examined over a concentration range of 10⁻⁶ to 10⁻⁸ M as shown in FIGS. 17 and 18. In contrast with the ATF-methioninase fusion protein, the TGF-methioninase fusion protein caused either no inhibition or a very small inhibition of cell proliferation and migration (significant inhibition of cell proliferation only at 10⁻⁶ M on day 3 and of cell migration only at 10⁻⁶ M on days 2-3, for p<0.05).

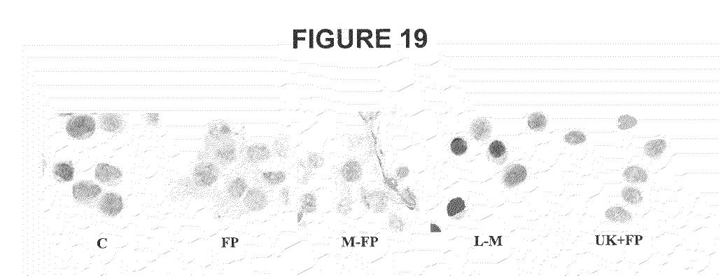

Specific Binding of ATF-methioninase Fusion Protein to MCF-7 Cells. In these experiments, the binding of the ATF-methioninase fusion protein to urokinase receptors on MCF-7 cells was demonstrated using a specific L-methioninase antibody for immunocytochemistry. As shown in FIG. 19, treatment of the cells with fusion protein (FIG. 19, FP) or mutated FP (FIG. 19, M-FP) resulted in a positive immuno-localization at the cell membrane, indicated by the red-brown staining of the antibody specific to L-methioninase. Immuno-localization was not observed with cells treated with fusion protein and urokinase, indicating a specific displacement of fusion protein by urokinase (FIG. 19, UK+FP). Further, immuno-localization was not observed for cells treated with L-methioninase alone (FIG. 19, L-M). All treatments were for 18 hours at a concentration of 10⁻⁶ M.

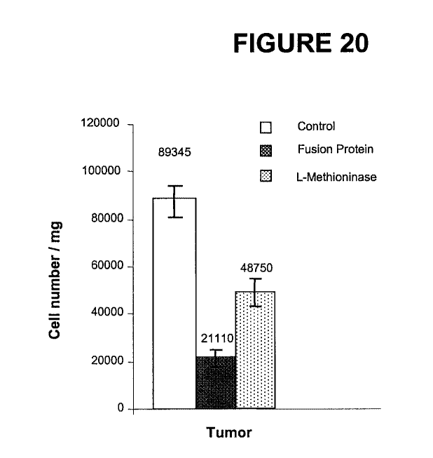

Effect of ATF-methioninase Fusion Protein on Mouse Xenografts. The nude mouse xenograft model was used to determine the influence of a 20-day ATF-methioninase fusion protein treatment on the growth of MCF-7 breast cancer cells in nude mouse xenografts (FIG. 20). The cell number/mg of tissue was quantified by measuring −gal activity in tissue homogenates. The results of this study demonstrated that the fusion protein was not cytotoxic to the nude mice during the 20-day treatment period. The fusion protein produced more than a 4-fold reduction in cancer cell number/mg tumor tissue as compared to the vehicle treated control group. Furthermore, fusion protein treatment was much more effective in reducing tumor growth than L-methioninase treatment. It was also observed that cancer cells in xenograft tumor sections from fusion protein and L-methioninase treated animals stained positively for L-methioninase by immunocytochemistry, while the control tumor sections stained negatively (data not shown).

### Discussion

The MCF-7 breast cancer cells used in the biological testing were previously verified to be methionine dependent In Example 1. Further, it has been shown in Example 1 that an ATF-methioninase fusion protein purified from E. coli inhibited MCF-7 cell proliferation and migration. Example 2 compared the effects of this same ATF-methioninase fusion protein to a methioninase-mutated fusion protein and L-methioninase alone. This fusion protein inhibited the migration and proliferation of MCF-7 breast cancer, PC-3 prostate cancer, and SK-LU-1 lung cancer cells over a concentration range of 10⁻⁶ to 10⁻⁸ M in a dose-dependent manner over a period of 3 days (FIGS. 11-16) and also inhibited the growth of MCF-7 cell xenografts in nude mice over 20 days (FIG. 20). This fusion protein was significantly more effective than free L-methioninase in inhibiting cell migration and proliferation, which supports our rationale for targeting L-methioninase to the surface of the cancer cells. Further, the lack of inhibition with the mutated fusion protein in this study demonstrates the effectiveness of active L-methioninase targeted to the cell surface.

Example 1 demonstrated that in a competitive binding assay, the ATF-methioninase fusion protein bound specifically to the urokinase receptor on MCF-7 cells in vitro, based on urokinase displacing the fusion protein from the membrane urokinase receptor. In Example 2, the binding specificity of this fusion protein to the urokinase receptor on the surface of MCF-7 cells was examined using immunocytochemistry employing a methioninase-specific antibody (FIG. 19). The results demonstrate that both fusion protein and mutated fusion protein bind to the surface of breast cancer cells (FIG. 19, FP and M-FP), while L-methioninase did not bind (FIG. 19, L-M). The fact that urokinase treatment eliminated fusion protein binding indicates the specificity of binding of the fusion protein to the urokinase receptor (FIG. 19, UK+FP). This also indicates that the use of a larger ATF in the fusion protein would possibly be more effective for binding to cancer cells that have the capability to produce urokinase, since it is known that the urokinase ATF 135 amino acids in length bind more strongly shorter ATF's (Appella et al., 1987). Therefore, the present invention also contemplates a fusion protein containing ATF₁₋₁₃₅ and L-methioninase.

The major mechanism of the inhibitory effects of ATF-methioninase is the methioninase-induced depletion of methionine available to the cells. The use of ATF to target L-methioninase to urokinase receptors on the surface of cancer cells is advantageous in the methods of the present invention because ATF is not internalized, since the catalytic domain of urokinase needed for internalization is not present (Cubellis et al., 1990). There are many different receptors on cancer cells, but nearly all of them cause internalization of their bound ligands and therefore would not be useful for targeting L-methioninase to the cell surface. This is clearly shown by the very minimal inhibition on cell proliferation and migration found for MCF-7 breast cancer cells treated with TGF-methioninase in the culture wounding assay (FIGS. 17 and 18). TGF-α has been shown previously to be internalized by the epidermal growth factor receptor on the cell surface (Korc et al., 1987), so it is expected that the TGF-methioninase fusion protein is internalized.

In Example 2, intra-tumoral injection was used to study the effect of ATF-methioninase on breast cancer xenografts in order to test the effect of delivering this fusion protein directly to the tumor, so that loss of the fusion protein by other, less direct means of delivery would not arise. There is, in fact, a growing interest in local intra-tumoral chemotherapy (see the review by Goldberg et al., 2002), so this is a definite possibility for the delivery of ATF-methioninase in a clinical setting. Intra-tumoral injection or infusion has extended to the delivery of proteins. For example, intra-tumoral infusion placed sterotactically in a malignant glioma was used to deliver a fusion protein consisting of IL-4 linked to P. aeruginosa exotoxin A (Rainov et al., 2004). This resulted in long-term survival of a patient for 3 years after fusion protein infusion with a durable tumor response. In another clinical application, 11 patients suffering from metastatic breast and colorectal cancers and from malignant melanomas were treated by intra-tumoral injection of a single-chain antibody-toxin fusion protein, with complete regression of tumor nodules in 40% of the patients and partial reduction in tumor size in 20% of the patients (Azemar et al., 2003).

For clinical delivery of ATF-methioninase, it may be necessary to reduce its immunogenicity, for example by conjugating it to polyethylene glycol (PEG). L-methioninase has been conjugated to PEG, resulting in a 36-fold increase in serum half-life and the elimination of immunogenic reactions (Yang et al., 2004). Therefore, PEGylation of ATF-methioninase or other conjugates described herein or that otherwise fall within the scope of the present invention are also contemplated in the methods of the present invention.

In summary, the ATF-methioninase fusion protein is significantly more effective in vitro and in vivo than free L-methioninase and appears to have potential as a therapeutic agent for cancer treatment.

### Example 3

### Binding of Recombinant L-Methioninase-Annexin V to Phosphatidyl Serine Immobilized on Plastic or on the Surface of Cancer Cells

In this example, the binding of annexin V, part of the fusion protein L-methioninase-annexin V, to phosphatidylserine (PS) by two different methods was demonstrated. In the first method, PS was immobilized on plastic microtiter plates. In the second method, PS was exposed on the surface of human breast cancer cells by the addition of hydrogen peroxide. Binding was measured using an antibody to L-methioninase. The proteins used in this study were produced by recombinant DNA technology in Escherichia Coli bacteria and were purified to homogeneity.

### Procedures for Preparation of Proteins

The protein genes were cloned into E. coli on the vector pET-30 Ek/LIC, which incorporates a His₆ tag at the N-terminus and an HRV 3C protease site just before the start of the desired protein. L-methioninase and annexin V are connected by the flexible linker Gly-Ser-Gly-Ser-Gly-Ser (SEQ ID NO:9) in the methioninase-annexin V fusion protein (SEQ ID NO:23, amino acid sequence thereof; SEQ ID NO:24, nucleotide sequence thereof). The sequences of the protein genes were verified by DNA sequencing.

Protein Expression: A culture of E. coli BL21(DE3) harboring pET-30 Ek/LIC with the fusion gene of interest (methioninase-annexin V, L-methioninase, or annexin V) was grown in 50 ml of LB medium containing 35 μg/ml ampicillin overnight at 37° C. with shaking. This cell culture was added to 1 liter of fresh culture medium, and the culture was grown with shaking at 37° C. This cell culture was grown to mid-log phase (OD₆₀₀=0.5), and protein expression was induced by adding isopropyl β-D-thiogalactopyranoside (IPTG) to a final concentration of 0.4 mM. After addition of IPTG, the shaking of cell culture was continued at 30° C. for 5 h and harvested by centrifugation for 10 min at 1000×g. The cell pellet was resuspended in 40 ml of sonication buffer containing 0.05 mM N-p-tosyl-L-phenylalanine chloromethyl ketone (TPCK), 1 mM phenylmethylsulfonyl fluoride (PMSF), 1% ethanol, 0.02 mM pyridoxal phosphate, 0.01%-mercaptoethanol and 0.02 M sodium phosphate at pH 7.4 (except ethanol and pyridoxal phosphate were not added for annexin V expression). The cells were lysed by sonication at 4° C. for 30 sec at 4.5 watts per ml of lysate and then allowed to cool for 30 sec on ice. This cycle was repeated for four times for a total sonication time of 2.5 min. The lysate obtained was centrifuged at 12,000×g for 30 min to remove the cell debris.

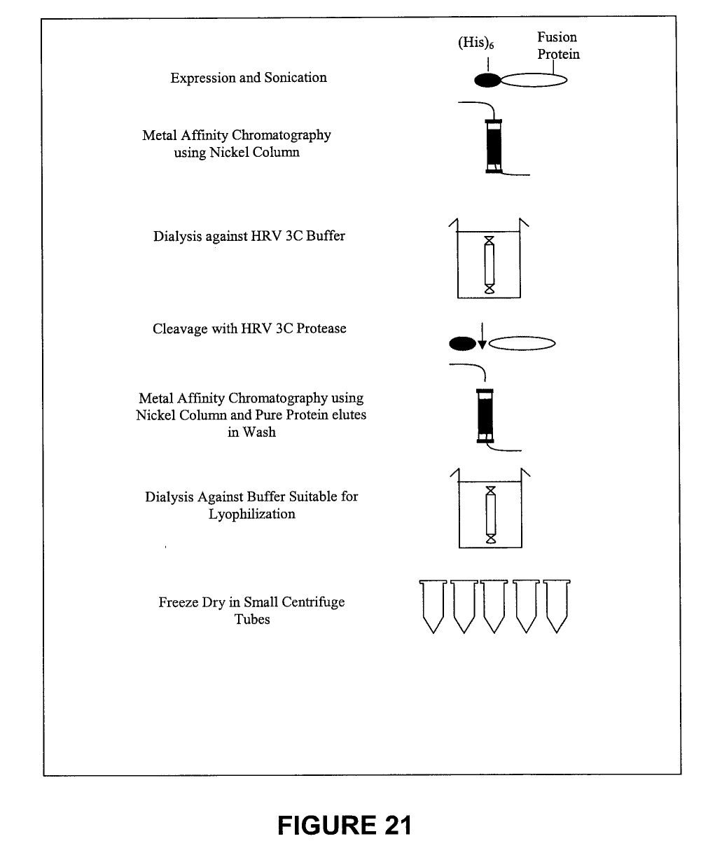

Protein Purification: All the purification steps were performed at 4° C. The entire purification procedure, outlined in FIG. 21, was performed as follows (except pyridoxal phosphate was not added for annexin V expression). Imidazole (40 mM) and NaCl (500 mM) were added to the lysate to reduce non-specific protein binding. This resulting mixture was fed to 5 ml HisTrap chromatography column, which was equilibrated with wash buffer containing 20 mM sodium phosphate, 40 mM imidazole, 500 mM NaCl, 0.02 mM pyridoxal phosphate at pH 7.4. The column was washed with the wash buffer to remove unwanted proteins. His-tagged fusion protein was eluted using elution buffer containing 20 mM sodium phosphate, 500 mM imidazole, 500 mM NaCl, 0.02 mM pyridoxal phosphate at pH 7.4. Eluted protein was dialyzed overnight against buffer containing 20 mM sodium phosphate, 0.02 mM pyridoxal phosphate at pH 7.4 to remove NaCl and imidazole from the protein solution and make suitable for N-terminal His-tag cleavage. The cleavage of N-terminal His-tag was achieved by using HRV 3C protease. HRV 3C protease (0.5 Units/mg of protein) and recommended 10× buffer (1.5 M NaCl, 0.5 M Tris-HCl, pH 7.5) were added to the protein solution. This reaction was carried out for 8 h at 4° C.

Imidazole (40 mM) and NaCl (500 mM) were added to the cleaved protein solution, which was fed again onto a 5 ml HisTrap column. Pure protein was eluted in a linear gradient of 0-0.5 M imidazole. Purified protein was dialyzed overnight against 20 mM sodium phosphate buffer at pH 7.4 containing 0.02 mM pyridoxal phosphate and 0.1 M NaCl.

Freeze Drying/Lyophilization: Purified protein obtained after chromatography was concentrated to the desired concentration using Centriprep centrifugal filters. Concentrated protein solution was then transferred to small 1.5 mL centrifuge tubes. A small pinhole was introduced on the cap of each tube to let vapors escape. These tubes were flash frozen using liquid nitrogen and transferred in 20 mL lyophilization vials holding 10-15 centrifuge tubes. Lyophilization vials were then loaded onto the freeze dryer for lyophilization. Typically, lyophilization was carried out for 8 h.

Methioninase Activity Assay: The enzymatic activity of L-methioninase was determined by measuring the absorbance of azine derivatives formed by reacting 3-methyl-2-benzothiazolone hydrazone hydrochloride and α-ketobutyrate (Esaki et al., 1987). α-ketobutyrate was the product obtained by enzymatic elimination of L-methionine by L-methioninase.

SDS-PAGE and Protein Assays: Cellular proteins and proteins after purification were separated on 12% (w/v) SDS-PAGE and stained with Coomassie brilliant blue. Protein concentration was measured by the Bradford assay.

### Procedures for Binding Tests

Binding of Methioninase-Annexin V on Plastic-immobilized Phosphatidylserine: Phosphatidylserine (PS) was dissolved in chloroform at a concentration of 50 μl/ml. This solution (100 μl) was added to wells of a 96-well polypropylene microtiter plates. This plate was allowed stay in a laminar flow hood until all the chloroform was evaporated. The plate was then blocked/washed by using phosphate buffered saline (PBS) solution containing 10% fetal bovine serum and 2 mM calcium chloride for 2 h at room temperature in the laminar flow hood. Methioninase-annexin V fusion protein was added to wash buffer at an initial concentration of 6.7 nM. Serial 2-fold dilutions of this concentrated fusion protein were done to give a final concentration of 6.7 μM. Methioninase-annexin V was added to wells in the increasing concentration of methioninase-annexin V. For each concentration of methioninase-annexin V, the experiment was done in triplets. After adding 100 μl of methioninase-annexin V, the plates were incubated for 2 h. The plates were washed with wash buffer, and primary antibody (rabbit anti-methioninase) diluted in wash buffer (1:1000) was added and incubated for 12 h at 4° C. The plates were again washed with wash buffer, and 100 μl of goat anti-rabbit IgG secondary antibody with HRP conjugated (1:1000 dilution in binding buffer) was added to the wells for 2 h at room temperature. The chromogenic substrate O-phenylenediamine (OPD, 200 μl) was used to detect the HRP by reading the plates at 450 nm.

Binding of Methioininase-Annexin V on Externally Positioned PS on the Surface of Cells: MCF-7 breast cancer cells were grown until they reached 85% confluence in T-75 flasks. Cancer cells (5×10⁴) were transferred to 24 well plates and grown until they reached 85% confluence. PS was exposed on the surface of cells by the addition of hydrogen peroxide (1 mM). Cells were treated with the DMEM media containing 1 mM of H₂O₂ for 1 h at 37° C. Cells were fixed with 0.25% glutaraldehyde diluted in PBS buffer containing Ca²⁺ (2 mM). Excess aldehyde groups were quenched by incubation with 50 mM of NH₄Cl for 5 min. Methioninase-annexin V fusion protein was added to wash buffer at an initial concentration of 6.7 nM. Serial 2-fold dilutions of this concentrated fusion protein solution were done to give a final concentration of 6.7 pM. Methioninase-annexin V was added to wells in the increasing concentration of methioninase-annexin V. For each concentration of methioninase-annexin, the experiment was done in triplets. After adding 300 μl of methioninase-annexin V, the plates were incubated for 2 h. The plates were washed with wash buffer, and primary antibody (rabbit anti-methioninase) diluted in wash buffer (1:1000) was added and incubated for 12 h at 4° C. The plates were again washed with wash buffer, and 300 μl of goat anti-rabbit IgG secondary antibody with HRP conjugated (1:1000 dilution in binding buffer) was added to the wells for 2 h at room temperature. After washing with PBS, the chromogenic substrate O-phenylenediamine (OPD, 300 μl) was added. After 30 min 100 μl of the supernatant was transferred to 96-well plates, and absorbance was measured at 450 nm.

### Results

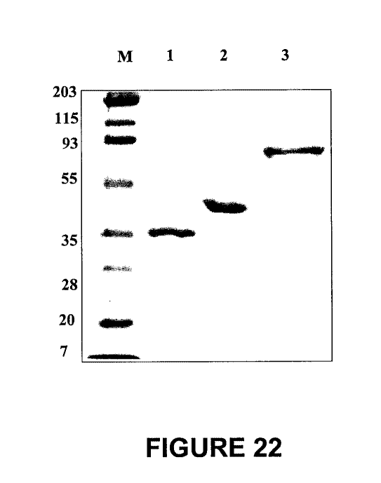

Protein Expression and Purification: An SDS-PAGE gel image showing the purified protein products of methioninase-annexin V, L-methioninase, and annexin V is shown in FIG. 22. It can be seen in FIG. 22 that the three proteins are highly pure. The yield and specific activity of the fusion proteins are shown in Table II. The specific activity results indicate that the L-methioninase activity on a molar basis is not reduced when it is linked to annexin V.

TABLE II
Yield and Specificity of Purified Proteins.

| | | Theoretical | | | |
|---|---|---|---|---|---|
| | | Molecular | | Yield | Specific |
| | Amino | Weight | | (mg/liter of | Activity |
| Protein | Acids | (kDa) | Runs | culture) | (Units/mg) |
| L-methioninase | 399 | 42.75 | 2 | 65.3 | 14.6 |
| Methioninase- | 729 | 79.62 | 1 | 41.3 | 8.6 |
| annexin V | | | | | |
| fusion protein | | | | | |
| Annexin V | 321 | 35.92 | 1 | 59.2 | 0 |

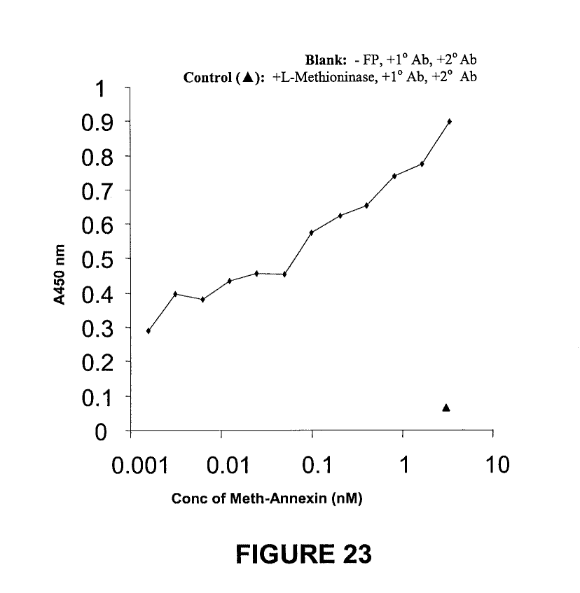

Binding of Methioninase-Annexin V on Plastic-immobilized Phosphatidylserine: The results in FIG. 23 show that the binding of methioninase-annexin V to PS immobilized on plastic plates increased as its concentration increased. The control result for L-methioninase was very low (A₄₅₀ less than 10% of that for methioninase-annexin V). These results confirm that methioninase-annexin V binds specifically to plastic-immobilized PS.

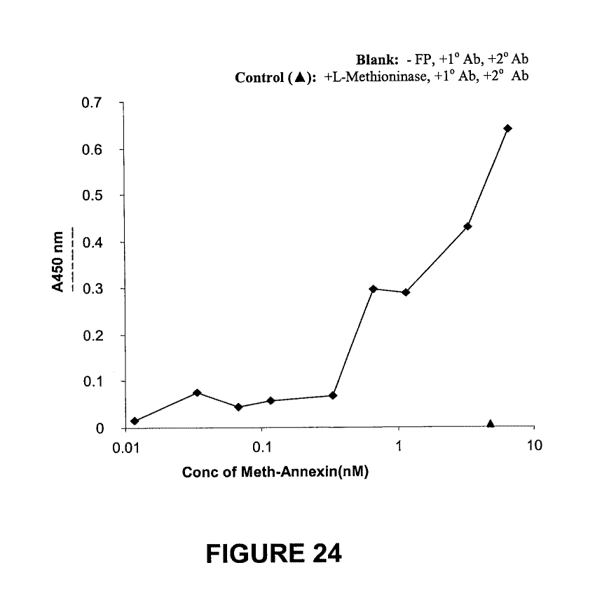

Binding of Methioninase-Annexin V on Externally Positioned PS on the Surface of MCF-7 Breast Cancer Cells: The binding of methioninase-annexin V to PS induced to be exposed on the cell surface was examined using MCF-7 breast cancer cells. FIG. 24 shows the binding of methioninase-annexin V to the MCF-7 breast cancer cells as its concentration is increased. The control result for L-methioninase showed negligible binding. These results confirm that methioninase-annexin binds to PS exposed on the MCF-7 breast cancer cells.

### Discussion

In this Example, it has been shown that the methioninase-annexin V fusion protein binds specifically to PS adsorbed on to plastic and on the membrane surface of MCF-7 breast cancer cells. Ran et al. obtained a similar result for the binding of annexin V to plastic-immobilized PS (Ran et al., 2002). All of the experiments were done in the presence of Ca²⁺ because it has been shown previously that binding of annexin V to PS is Ca²⁺ dependent. PS is attractive as a tumor vessel target for several reasons: it is abundant (PS is present at >10⁶ molecules per cell); it is on the luminal surface of tumor endothelium, which is directly accessible for binding by vascular targeting agents in the blood; it is present on a significant percentage of tumor vascular endothelial cells in diverse solid tumors; and it appears to be absent from vascular endothelium in all of the normal tissues.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents and peptides which are both chemically and physiologically related may be substituted for the agents and peptides described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abuchowski, A, van Es T, Palczuk, N C, Davis, F F (1977) Alteration of immunological properties of bovine serum albumin by covalent attachment of polyethylene glycol. J Biol Chem 252: 3578-3581.
Appella, E., Robinson, E. A., Ullrich, S. J., Stoppelli, M. P., Corti A., Cassani, G., Blasi, F. (1987) The receptor-binding sequence of urokinase. J. Biol. Chem. 262: 4437-4440.
Argos, P. (1990) An investigation of oligopeptides linking domains in protein tertiary structures and possible candidates for general gene fusion. J. Mol. Biol. 211: 943-958.
Arteaga, C. L., Hurd, S. D., Dugger, T. C., Winnier, A. R., and Robertson, J. B. (1994). Epidermal growth factor receptors in human breast carcinoma cells: a potential selective target for transforming growth factor-Pseudomonas exotoxin 40 fusion protein. Cancer Res. 54: 4703-4709.
Ausubel, F. M., Brent, R., Kingston, R. E., Moore, D. D., Seidman, J. G., Smith, J. A., and Struhl, K. (1994-1997) Current Protocols in Molecular Biology, Wiley, New York.
Avramis, V. I., Sencer. S., Periclou. A. P., Sather, H., Bostrom, B. C., Cohen, L. J., Ettinger, A. G., Ettinger, L. J., Franklin, J., Gaynbon, P. S., Hilden, J. M., Lange, B., Majlessipour, F., Mathew, P., Needle, M., Neglia, J., Reaman, G., Holcenberg, J. S., and Stork, L. (2002) A randomized comparison of native Escherichia coli asparaginase and polyethylene glycol conjugated asparaginase for treatment of children with newly diagnosed standard-risk acute lymphoblastic leukemia: a Children's Cancer Group study. Blood 99: 1986-1994.
Azemar, M., Djahansouzi, S., Jager, E., Solbach, C., Schmidt, M., Maurer, A. B., Mross, K., Unger, C., von Minckwitz, G., Dall, P., Groner, B., and Wels, W. S. (2003) Regression of cutaneous tumor lesions in patients intratumorally injected with a recombinant single-chain antibody-toxin targeted to ErbB2/HER2. Breast Cancer Res Treat, 82: 155-164.
Bartucci, M., Morelli, C., Mauro, L., Ando, S., and Surmacz, E. (2001) Differential insulin-like growth factor I receptor signaling and function in estrogen receptor (ER)-positive MCF-7 and ER-negative MDA-MB-231 breast cancer cells. Cancer Res, 61: 6747-6754.
Batra, J. K., Jinno, Y., Chaudhary, V. K., Kondo, T., Willingham, M. C., FitzGerald, D. J., and Pastan, I. (1989) Antitumor activity in mice of an immunotoxin made with anti-transferrin receptor and a recombinant form of Pseudomonas exotoxin. Proc. Natl. Acad. Sci. USA 86: 8545-8549.
Buller, C. J., Zang, X. P., Howard, E. W., and Pento, J. T. (2003) Measurement of beta-galactosidase tissue levels in a tumor cell xenograft model. Methods Find Exp Clin Pharmacol, 25: 713-716.
Cavallaro, U., del Vecchio, A., Lappi, D. A., Soria, M. R. (1993) A conjugate between human urokinase and saporin, a type-1 ribosome-inactivating protein, is selectively cytotoxic to urokinase receptor-expressing cells. J. Biol. Chem. 268: 23186-23190.
Chen, L., Shulman, L. M., and Revel, M. (1991) IL-6 receptors and sensitivity to growth inhibition by IL-6 in clones of human breast carcinoma cells. J Biol Regul. Homeost. Agents, 5: 125-136.
Concha, N. O., Head, J. F., Kaetzel, M. A., Dedman, J. R., and Seaton, B. A. (1993) Rat annexin V crystal structure: Ca²⁺-induced conformational changes. Science, 261:1321-1324.
Cubellis, M. V., Wun, T. C., and Blasi, F. (1990) Receptor-mediated internalization and degradation of urokinase is caused by its specific inhibitor PAI-1. Embo J, 9: 1079-1085.
Deng, W. P., and Nickoloff, J. A. (1992) Site-directed mutagenesis of virtually any plasmid by eliminating a unique site. Anal. Biochem. 200: 81-88.
Drug Information for L-Asparaginase, Jan. 5, 2003, www.mdbrouse.com.
Ellis, V., Pyke, C., Eriksen, J., Solberg, H., Dano, K. (1992) The urokinase receptor: involvement in cell surface proteolysis and cancer invasion. Ann. N.Y. Acad. Sci. 667: 13-31.
Esaki, N., and Soda, K. (1973) L-methionine-lyase from Pseudomonas putida and aeromonas. Meth. Enzymol. 143: 459-465.
Gaspar, M. M., Perez-Soler, R., and Cruz, M. E. M. (1996) Biological characterization of L-asparaginase liposomal formulations. Cancer. Chemother. Pharmacol. 38: 373-377.
Goldberg, E. P., Hadba, A. R., Almond, B. A., and Marotta, J. S. (2002) Intratumoral cancer chemotherapy and immunotherapy: opportunities for nonsystemic preoperative drug delivery. J Pharm Pharmacol, 54: 159-180.
Gooch, J. L., Lee, A. V., and Yee, D. (1998) Interieukin 4 inhibits growth and induces apoptosis in human breast cancer cells. Cancer Res, 58: 4199-4205.
Halpern, B. C., Clark, B. R., Hardy, D. N., Halpern, R. M., and Smith, R. A. (1974) The effect of replacement of methionine by homocystine on survival of malignant and normal adult mammalian cells in culture. Proc. Nat. Acad. Sci. USA 71: 1133-1136.
Hellman, S, and Vokes, E. E. (1996) Advancing current treatments for cancer. Sci. Amer. 275:118-123.
Hori, H., Takabayashi, K., Orvis, L., Carson, D. A., and Nobori, T. (1996) Gene cloning and characterization of Pseudomonas putida L-methionine-1-deamino-1-mercaptomethane-lyase. Cancer Res. 56: 2116-2122.
Howard, M., Farrar, J., Hilfiker, M., Johnson, B., Takatsu, K., Hamaoka, T., and Paul, W. E. (1982) Identification of a T cell-derived b cell growth factor distinct from interleukin 2. J Exp Med, 155: 914-923.
Huber, R., Romisch, J., and Paques, E. P. (1990) The crystal and molecular structure of human annexin V, an anticoagulant protein that binds to calcium and membranes. Embo J., 3867-3874.
Inoue, H., Inagaki, K., Sugimoto, M., Esaki, N., Soda, K., and Tanaka, H. (1995) Structural analysis of the L-methionine-lyase gene from Pseudomonas putida. J. Biochem. 117: 1120-1125.
Inoue, H., Inagaki, K., Adachi, N., Tamura, T., Esaki, N., Soda, K., and Tanaka, H. (2000) Role of tyrosine 114 of L-methionine gamma-lyase from Pseudomonas putida. Biosci Biotechnol Biochem, 64: 2336-2343.
Jacobs, P., Cravador, A., Loriau, R., Brockly, F., Colau, B., Chuchana, P., Van Elsen, A., Herzog, A., and Bollen, A. (1985) Molecular cloning, sequencing, and expression in Escherichia coli of human preprourokinase cDNA. DNA 4: 139-146.
Jain, P. T., Rajah, T. T., and Pento, J. T. (1997) Antitumor activity of a novel antiestrogen (Analog II) on human breast cancer cells. Anti-Cancer Drugs 8: 964-975.
Jemel, A., Thomas, A., Murray, T., and Thun, M. (2002) Cancer statistics 2002. Cancer J. Clin. 52: 23-47.
Koivunen et al. (1999) Tumor targeting with a selective gelatinase inhibitor. Nature Biotechnology, 17:768-774.
Kokkinakis, D. M., von Wronski, M. A., Vuong, T. H., Brent, T. P., and Schold, S. C. (1997) Regulation of 06-methylguanine-DNA methyltransferase by methionine in human tumor cells. Br. J. Cancer 75: 779-788.
Kokkinakis, D. M., Schold, S. C., Hori, H., and Nobori, T. (1997) Effect of long-term depletion of plasma methionine on the growth and survival of human brain tumor xenografts in athymic mice. Nutrition and Cancer 29: 195-204.
Korc, M., Haussler, C. A., and Trookman, N. S. (1987) Divergent effects of epidermal growth factor and transforming growth factors on a human endometrial carcinoma cell line. Cancer Res, 47: 4909-4914.
Laemmli, U. K. (1970) Cleavage of structural proteins during assembly of the head of bacteriophage T4. Nature (Lond) 227: 680-685.
Li, Y., Wood, N., Yellowlees, D., and Donnelly, P. K. (1999) Cell surface expression of urokinase receptor in normal mammary epithelial cells and breast cancer cell lines. Anticancer Res, 19: 1223-1228.
Lilie, H., Schwarz, E., Rudolph, R. (1998) Advances in refolding of proteins produced in E. coli. Curr. Opin. Biotechnol. 9: 497-501.
Marquardt, H., Hunkapiler, M. W., Hood, L. E., and Todaro, G. (1984) Rat transforming growth factor type I: structure and relation to epidermal growth factor. Science 223: 1979-1081.
McKie, A. E., Edlind, T., Walker, J., Mottram, J. C., and Coombs, G. H. (1998) The primitive protozoon Trichomonas vaginalis contains two methionine-lyase genes that encode members of the -family of pyridoxal 5′-phosphate-dependent enzymes. J. Biol. Chem. 273: 5549-5556.
Mecham, J. C., Rowitch, D., Wallace, C. D., Stern, P. H., and Hoffman, R. M. (1983) The metabolic defect of methionine dependence occurs frequently in human tumor cell lines. Biochem. Biophys. Res. Commun. 117: 429-434.
Miki, K., Al-Refaie, W., Xu, M., Jiang, P., Tan, Y., Bouvet, M., Zhao, M., Gupta, A., Chishima, T., Shimada, H., Makuuchi, M., Moossa, A. R., and Hoffman, R. M. (2000) Methioninase gene therapy of human cancer cells is synergistic with recombinant methioninase treatment. Cancer Res. 60: 2696-2702.
Mickisch, G. H., Pai, L. H., Siegsmund, M., Campain, J., Gottesman, M. M. and Pastan, I. (1993) Pseudomonas exotoxin conjugated to monoclonal antibody MRK16 specifically kills multidrug resistant cells in cultured renal carcinomas and in MDR-transgenic mice. J. Urology 149: 174-178.
Muraguchi, A., Hirano, T., Tang, B., Matsuda, T., Horii, Y., Nakajima, K., and Kishimoto, T. The essential role of B cell stimulatory factor 2 (BSF-2/IL-6) for the terminal differentiation of B cells. J Exp Med, 167: 332-344, 1988.
Nakayama, T., Esaki, N., Sugie, K., Beresov, T. T., Tanaka, H., and Soda, K. (1984) Purification of bacterial L-methionine-lyase. Anal. Biochem. 138: 421-424.
Nguyen, D. H. D., Hussaini, I. M., and Gonias, S. L. (1998) Binding of urokinase-type plasminogen activator to its receptor in MCF-7 cells activates extracellular signal-regulated kinase 1 and 2 which is required for increased cellular motility. J. Biol. Chem. 273: 8502-8507.
Nguyen, T. T., Zang, X. P., Pento, J. T. (2002) Effects of KGF inhibitors on the migration and proliferation of breast cancer cells in a culture wounding model. Pharmacol. Res. 46: 79-83.
Nilsson, F., Kosmehl, H., Zardi, L., and Neri, D. (2001) Targeted delivery of tissue factor to the ED-B domain of fibronectin, a marker of angiogenesis, mediates the infarction of soluid tumors in mice. Cancer Res. 61: 711-716.
Pastan, I., Chaudhary, V., and FitzGerald, D. J. (1992) Recombinant toxins as novel therapeutic agents. Annu. Rev. Biochem. 61: 331-354.
Peron, K., Jones, T. N., Gauthier, S. A., Nguyen, T. T., Zang, X. P., Barriere, M., Preveraud, D., Soliman, C. E., Harrison, R. G., and Pento, J. T. (2003) Targeting of a novel fusion protein containing methioninase to the urokinase receptors to inhibit breast cancer cell migration and proliferation. Cancer. Chemother. Pharmacol. 52: 270-276.
Phillips, P. C., Levow, C., Catterall, M., Colvin, M. C., Pastan, I., and Brem, H. (1994) Transforming growth factor-—Pseudomonas exotoxin fusion protein (TGF-1-PE38) treatment of subcutaneous and intracranial human glioma and medulloblastoma xenografts in athymic mice. Cancer Res. 54: 1008-1015.
Prior, T. I., Helman, L. J., FitzGerald, D. J., and Pastan, I. Cytotoxic activity of a recombinant fusion protein between insulin-like growth factor I and Pseudomonas exotoxin. Cancer Res, 51: 174-180, 1991.
Pyke, C., Kristensen, P., Ralfkiaer, E., Grondahl-Hansen, J., Eriksen, J., Blasi, F., and Dano, K. (1991) Urokinase-type plasminogen activator is expressed in stromal cells and its receptor in cancer cells at invasive foci in human colon adenocarcinomas. Am J Pathol, 138: 1059-1067.
Rabbani, S. A., Mazar, A. P., Bernier, S. M., Haq, M., Bolivar. I. (1992) Structural requirements for the growth factor activity of the amino-terminal domain of urokinase. J Biol Chem 267: 14141-14156.
Rainov, N. G. and Heidecke, V. (2004) Long term survival in a patient with recurrent malignant glioma treated with intratumoral infusion of an IL4-targeted toxin (NBI-3001). J Neurooncol, 66: 197-201.
Rajah, T. T., and Pento, J. T. (1998) Influence of antiestrogens on the invasiveness and laminin attachment of MDA-MB-231 breast cancer cells. Cancer Invest., 17: 10-18.
Ran, S., Downes, A. and Thorpe, P. E. (2002a) Increased exposure of anionic phospholipids on the surface of tumor blood vessels. Cancer Res, 62:6132-40.
Ran, S., and Thorpe, P. E. (2002b) Phosphatidylserine is a marker of tumor vasculature and a potential target for cancer imaging and therapy. Int. J. Radiation Oncology Biol. Phys., 54:1479-1484.
Rao, L. V. M., Tait, J. F., and Hoang, A. D. (1992) Binding of annexin V to a human ovarian carcinoma cell line (OC-2008). Contrasting effects on cell surface factor Vila/tissue factor activity and prothrombinase activity. Thromb. Res. 67: 517-531.
Ruoslahti, E. (2004) Vascular zip codes in antiogenesis and metastasis. Biochemical Society Transactions, 32:397-402.
Sambroook, J., Fritsch, E. F., and Maniatis, T. (1989) Molecular Cloning: A Laboratory Manual, Cold Spring Harbor Laboratory Press, Cold Spring Harbor, N.Y.
Schein, C. H., and Noteborn, M. H. M. (1988) Formation of soluble recombinant proteins in Escherichia coli is favored by lower growth temperature. Bio/Technol. 6: 291-293.
Stephens, R. W., Bokman, A. M., Myohanen, H. T., Reisberg, T., Tapiovaara, H., Pedersen, N., Grondahl-Hansen, J., Llinas, M., and Vaheri, A. (1992) Heparin binding to the urokinase kringle domain. Biochemistry 31: 7572-7579.
Stoppelli, M. P., Corti, A., Soffientini, A., Cassani, G., Blasi, F., and Assoian, R. K. (1985) Differentiation-enhanced binding of the amino-terminal fragment of human urokinase plasminogen activator to a specific receptor on U937 monocytes. Proc. Natl. Acad. Sci. USA 82: 4939-4943.
Sugimura, et al., (1994) “Blood Coagulation”, Fibrinolysis, 5:365-373.
Tait, J. F., Engelhardt, S., Smith, C., and Fujikawa, K. (1995) Prourokinase-annexin V chimeras. Construction, expression, and characterization of recombinant proteins. J. Biol. Chem., 270:21594-21599.
Tan, Y., Zavala, J., Xu, M., Zavala Jr., J., Hoffman, R. M. (1996) Serum methionine depletion without side effects by methioninase in metastatic breast cancer patients. Anticancer Res. 16: 3937.
Tan, Y., Zavala, J., Sr., Han, Q., Xu, M., Sun, X., Tan, X., Magana, R., Geller, J., and Hoffman, R. M. (1996) Recombinant methioninase infusion reduces the biochemical endpoint of serum methionine with minimal toxicity in high-stage cancer patients. Anticancer Res, 17: 3857-3860.
Tan, Y., Sun, X., Xu, M., An, Z., Tan, X., Han, Q., Miljkovic, D. A., Yang, M., and Hoffman, R. M. (1998) Polyethylene glycol conjugation of recombinant methioninase for cancer therapy. Protein Expr. Purif. 12: 45-52.
Utsugi, T., Schroit, A. J., Connor J. et al. (1991) Elevated expression of phosphatidylserine in the outer membrane leaflet of human tumor cells and recognition by activated human blood monocytes. Cancer Res. 51: 3062-3066.
Vallera, D. A., Li, C., Jin, N., Panoskaltsis-Mortari, A., and Hall, W. A. (2002) Targeting urokinase-type plasminogen activator receptor on human glioblastoma tumors with diphtheria toxin fusion protein DTAT. J. Nat. Cancer Inst. 17: 597-606.
Voges, D., Berendes, R., Burger, A., Demange, P., Baumeister, W., and Huber, R. (1994) Three-dimensional structure of membrane-bound annexin V. A correlative electron microscopy-X-ray crystallography study. J. Mol. Biol., 238:199-213.
Xu et al. (1997) Endothelial and macrophage upregulation of urokinase receptor expression in human renal cell carcinoma. Human Pathology, 29:206-213.
Yamamoto, M., Sawaya, R., Mohanam, S., Rao, V. H., Bruner, J. M., Nicolson, G. L., and Rao, J. S. (1994) Expression and localization of urokinase-type plasminogen activator receptor in human gliomas. Cancer Res, 54: 5016-5020.
Yang, Z., Wang, J., Lu, Q., Xu, J., Kobayashi, Y., Takakura, T., Takimoto, A., Yoshioka, T., Lian, C., Chen, C., Zhang, D., Zhang, Y., Li, S., Sun, X., Tan, Y., Yagi, S., Frenkel, E. P., and Hoffman, R. M. (2004) PEGylation confers greatly extended half-life and attenuated immunogenicity to recombinant methioninase in primates. Cancer Res, 64: 6673-6678.
Ylikangas, P., and Mononen, I. (2000) A fluorometric assay for L-asparaginase activity and monitoring of L-asparaginase therapy. Anal. Biochem. 280: 43-45.
Zang, X. P., Lerner, M. R., Dunn, S. T., Brackeft, D. J., and Pento, J. T. (2003) Antisense KGFR oligonucleotide inhibition of KGF-induced motility in breast cancer cells. Anticancer Res, 23: 4913-4919.
Zang, X. P., Palwai, N. R., Pento, J. T., and Harrison, R. G. (2006) Internalizing versus non-internalizing receptors for targeting L-methioninase to cancer cells. Amer J Pharmacol Toxicol. 1:60-64.

What is claimed is:

1. A method of treating a cancer tumor or cancer cells wherein the cancer tumor or cancer cells is supplied by a tumor vasculature, comprising the step of:
providing a conjugate comprising a ligand having L-methioninase operatively attached thereto, wherein the ligand has the ability to specifically and stably bind to an external receptor or binding site present on an outer surface of a tumor vasculature endothelial cell and wherein the external receptor or binding site is specific for tumor vasculature endothelial cells;
contacting at least one blood vessel supplying a tumor with a therapeutically effective amount of the conjugate, whereby the conjugate is maintained on the outer surface of the tumor vasculature endothelial cell with substantially no internalization of the conjugate, and wherein the L-methioninase is stably bound to the outer surface of the tumor vasculature endothelial cell such that exogenous methionine in close proximity to the tumor vasculature endothelial cell is degraded and thus not delivered via the tumor vasculature to the cancer tumor or cancer cells, whereby the conjugate is selectively toxic to cancer cells being supplied by the at least one blood vessel.


SEQUENCE LISTING (28 sequences)

SEQ ID NO: 1 - protein, 452 aa, artificial sequence
  fusion protein comprising N-(amino acids 1-49 of urokinase A chain)-Gly-Ser-Gly-Ser-Gly-Ser-(L-methioninase from Pseudomonas putida)-C
SNELHQVPSNCDCLNGGTCVSNKYFSNIHWCNCPKKFGGQHCEIDKSKTGSGSGSRDSHNNTGFSTRAIHHGYDPLSHGGALVPPVYQTATYAFPTVEYGAACFAGEEAGHFYSRISNPTLALLEQRMASLEGGEAGLALASGMGAITSTLWTLLRPGDELIVGRTLYGCTFAFLHHGIGEFGVKIHHVDLNDAKALKAAINSKTRMIYFETPANPNMQLVDIAAVVEAVRGSDVLVVVDNTYCTPYLQRPLELGADLVVHSATKYLSGHGDITAGLVVGRKALVDRIRLEGLKDMTGAALSPHDAALLMRGIKTLALRMDRHCANALEVAQFLAGQPQVELIHYPGLPSFAQYELAQRQMRLPGGMIAFELKGGIEAGRGFMNALQLFARAVSLGDAESLAQHPASMTHSSYTPQERAHHGISEGLVRLSVGLEDVEDLLADIELALEACA

SEQ ID NO: 2 - DNA, 1362 bp, artificial sequence
  DNA sequence of the fusion protein of SEQ ID NO1
atgagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaaccggcagcggttctggatcccgcgactcccataacaacaccggtttttccacacgggccattcaccacggctacgacccgctttcccacggtggtgccttggtgccaccggtgtaccagaccgcgacctatgccttcccgactgtcgaatacggcgctgcgtgcttcgccggggaggaggcggggcacttctacagccgcatctccaaccccaccctggccttgctcgagcaacgcatggcctcgttggagggtggtgaggcgggattggcgctggcgtcggggatgggagccattacttcgaccctctggaccctgctgcggcctggtgatgagctgatcgtggggcgcaccttgtatggctgcacctttgcgttcctgcaccatggcattggcgagttcggggtcaagatccaccatgtcgaccttaacgatgccaaggccctgaaagcggcgatcaacagcaaaacgcggatgatctacttcgaaacaccggccaaccccaacatgcaactggtggatatagcggcggtcgtcgaggcagtgcgggggagtgatgtgcttgtggtggtcgacaacacctactgcacgccctacctgcagcggccactggaactgggggcagacctggtggtgcattcggcaaccaagtacctcagtggccatggcgacatcactgcgggcctggtggtggggcgcaaggctttggtcgaccgcattcggctggaagggctgaaagacatgaccggggcagccttgtcaccgcatgacgctgcgttgttgatgcgcggcatcaagaccctggcgctgcgcatggaccggcattgcgccaacgccctggaggtcgcgcagttcctggccgggcagccccaggtggagctgatccactacccgggcttgccgtcgtttgcccagtacgaactggcacagcggcagatgcgtttgccgggcgggatgattgcctttgagctcaagggcggtatcgaggccgggcgcggcttcatgaatgccctgcagctttttgcccgtgcggtgagcctgggggatgccgagtcgctggcacagcacccggcgagcatgacgcactccagttacacgccacaagagcgggcgcatcacgggatatcagaggggctggtgaggttgtcagtggggctggaggatgtggaggacctgctggcagatatcgagttggcgttggaggcgtgtgcatga

SEQ ID NO: 3 - protein, 49 aa, Homo sapiens
SNELHQVPSNCDCLNGGTCVSNKYFSNIHWCNCPKKFGGQHCEIDKSKT

SEQ ID NO: 4 - DNA, 147 bp, Homo sapiens
agcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacc

SEQ ID NO: 5 - protein, 6 aa, artificial sequence
  linker sequence
GSGSGS

SEQ ID NO: 6 - DNA, 18 bp, artificial sequence
  linker sequence
ggcagcggttctggatcc

SEQ ID NO: 7 - protein, 398 aa, Pseudomonas putida
MRDSHNNTGFSTRAIHHGYDPLSHGGALVPPVYQTATYAFPTVEYGAACFAGEEAGHFYSRISNPTLALLEQRMASLEGGEAGLALASGMGAITSTLWTLLRPGDELIVGRTLYGCTFAFLHHGIGEFGVKIHHVDLNDAKALKAAINSKTRMIYFETPANPNMQLVDIAAVVEAVRGSDVLVVVDNTYCTPYLQRPLELGADLVVHSATKYLSGHGDITAGLVVGRKALVDRIRLEGLKDMTGAALSPHDAALLMRGIKTLALRMDRHCANALEVAQFLAGQPQVELIHYPGLPSFAQYELAQRQMRLPGGMIAFELKGGIEAGRGFMNALQLFARAVSLGDAESLAQHPASMTHSSYTPQERAHHGISEGLVRLSVGLEDVEDLLADIELALEACA

SEQ ID NO: 8 - DNA, 1197 bp, Pseudomonas putida
atgcgcgactcccataacaacaccggtttttccacacgggccattcaccacggctacgacccgctttcccacggtggtgccttggtgccaccggtgtaccagaccgcgacctatgccttcccgactgtcgaatacggcgctgcgtgcttcgccggggaggaggcggggcacttctacagccgcatctccaaccccaccctggccttgctcgagcaacgcatggcctcgttggagggtggtgaggcgggattggcgctggcgtcggggatgggagccattacttcgaccctctggaccctgctgcggcctggtgatgagctgatcgtggggcgcaccttgtatggctgcacctttgcgttcctgcaccatggcattggcgagttcggggtcaagatccaccatgtcgaccttaacgatgccaaggccctgaaagcggcgatcaacagcaaaacgcggatgatctacttcgaaacaccggccaaccccaacatgcaactggtggatatagcggcggtcgtcgaggcagtgcgggggagtgatgtgcttgtggtggtcgacaacacctactgcacgccctacctgcagcggccactggaactgggggcagacctggtggtgcattcggcaaccaagtacctcagtggccatggcgacatcactgcgggcctggtggtggggcgcaaggctttggtcgaccgcattcggctggaagggctgaaagacatgaccggggcagccttgtcaccgcatgacgctgcgttgttgatgcgcggcatcaagaccctggcgctgcgcatggaccggcattgcgccaacgccctggaggtcgcgcagttcctggccgggcagccccaggtggagctgatccactacccgggcttgccgtcgtttgcccagtacgaactggcacagcggcagatgcgtttgccgggcgggatgattgcctttgagctcaagggcggtatcgaggccgggcgcggcttcatgaatgccctgcagctttttgcccgtgcggtgagcctgggggatgccgagtcgctggcacagcacccggcgagcatgacgcactccagttacacgccacaagagcgggcgcatcacgggatatcagaggggctggtgaggttgtcagtggggctggaggatgtggaggacctgctggcagatatcgagttggcgttggaggcgtgtgcatga

SEQ ID NO: 9 - protein, 5 aa, Artificial sequence
  linker for fusion protein
GSGSG

SEQ ID NO: 10 - protein, 8 aa, Homo sapiens
SNELHQVP

SEQ ID NO: 11 - protein, 21 aa, Homo sapiens
DCLNGGTCVSNKYFSNIHWCN

SEQ ID NO: 12 - DNA, 62 bp, Artificial Sequence
  Primer for construction of ATF-methioninase fusion protein
gacgacgacaagatgcttgaagtcctctttcagggacccagcaatgaacttcatcaagttcc

SEQ ID NO: 13 - DNA, 45 bp, Artificial Sequence
  Primer for construction of ATF-methioninase fusion protein
gaggagaagcccggttatcatgcacacgcctccaatgccaactcg

SEQ ID NO: 14 - DNA, 45 bp, Artificial Sequence; identical to SEQ ID NO: 13
  Primer for construction of ATF-methioninase fusion protein

SEQ ID NO: 15 - protein, 455 aa, Artificial Sequence
  fusion protein of N-(TGF-alpha)-Gly-Ser-Gly- Ser-Gly-Ser-(L-methioninase from Pseudomonas putida)-C
GPVVSHFNDCPDSHTQFCFHGTCRFLVQEDKPACVCHSGYVGARCEHADLLAGSGSGSRDSHNNTGFSTRAIHHGYDPLSHGGALVPPVYQTATYAFPTVEYGAACFAGEEAGHFYSRISNPTLALLEQRMASLEGGEAGLALASGMGAITSTLWTLLRPGDELIVGRTLYGCTFAFLHHGIGEFGVKIHHVDLNDAKALKAAINSKTRMIYFETPANPNMQLVDIAAVVEAVRGSDVLVVVDNTYCTPYLQRPLELGADLVVHSATKYLSGHGDITAGLVVGRKALVDRIRLEGLKDMTGAALSPHDAALLMRGIKTLALRMDRHCANALEVAQFLAGQPQVELIHYPGLPSFAQYELAQRQMRLPGGMIAFELKGGIEAGRGFMNALQLFARAVSLGDAESLAQHPASMTHSSYTPQERAHHGISEGLVRLSVGLEDVEDLLADIELALEACA

SEQ ID NO: 16 - DNA, 1365 bp, Artificial Sequence
  DNA sequence of fusion protein of SEQ ID NO15
ggacccgtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccggcagcggttctggatcccgcgactcccataacaacaccggtttttccacacgggccattcaccacggctacgacccgctttcccacggtggtgccttggtgccaccggtgtaccagaccgcgacctatgccttcccgactgtcgaatacggcgctgcgtgcttcgccggggaggaggcggggcacttctacagccgcatctccaaccccaccctggccttgctcgagcaacgcatggcctcgttggagggtggtgaggcgggattggcgctggcgtcggggatgggagccattacttcgaccctctggaccctgctgcggcctggtgatgagctgatcgtggggcgcaccttgtatggctgcacctttgcgttcctgcaccatggcattggcgagttcggggtcaagatccaccatgtcgaccttaacgatgccaaggccctgaaagcggcgatcaacagcaaaacgcggatgatctacttcgaaacaccggccaaccccaacatgcaactggtggatatagcggcggtcgtcgaggcagtgcgggggagtgatgtgcttgtggtggtcgacaacacctactgcacgccctacctgcagcggccactggaactgggggcagacctggtggtgcattcggcaaccaagtacctcagtggccatggcgacatcactgcgggcctggtggtggggcgcaaggctttggtcgaccgcattcggctggaagggctgaaagacatgaccggggcagccttgtcaccgcatgacgctgcgttgttgatgcgcggcatcaagaccctggcgctgcgcatggaccggcattgcgccaacgccctggaggtcgcgcagttcctggccgggcagccccaggtggagctgatccactacccgggcttgccgtcgtttgcccagtacgaactggcacagcggcagatgcgtttgccgggcgggatgattgcctttgagctcaagggcggtatcgaggccgggcgcggcttcatgaatgccctgcagctttttgcccgtgcggtgagcctgggggatgccgagtcgctggcacagcacccggcgagcatgacgcactccagttacacgccacaagagcgggcgcatcacgggatatcagaggggctggtgaggttgtcagtggggctggaggatgtggaggacctgctggcagatatcgagttggcgttggaggcgtgtgca

SEQ ID NO: 17 - DNA, 68 bp, Artificial Sequence
  primer for PCR for TGF-methioninase of SEQ ID NOS15/16
gacgacgacaagatgcttgaagtcctctttcagggacccggagtggtgtcccattttaatgactgccc

SEQ ID NO: 18 - DNA, 47 bp, Artificial Sequence
  Primer for PCR for TGF-methioninase of SEQ ID NOS15/16
gcggatccagaaccgctgccagccaggaggtccgcatgctcacagcg

SEQ ID NO: 19 - DNA, 30 bp, Artificial Sequence
  Primer for PCR for TGF-methioninase of SEQ ID NOS15/16
cgcggatcccgcgactcccataacaacacc

SEQ ID NO: 20 - DNA, 45 bp, Artificial Sequence; identical to SEQ ID NO: 13
  Primer for PCR for TGF-methioninase of SEQ ID NOS15/16

SEQ ID NO: 21 - DNA, 26 bp, Artificial Sequence
  Primer for site-directed mutagenesis of ATF-methioninase
gcgcaccttgtttggctgcacctttg

SEQ ID NO: 22 - DNA, 26 bp, Artificial Sequence
  Primer for site directed mutagenesis of ATF-methioninase
caaaggtgcagccaaacaaggtgcgc

SEQ ID NO: 23 - protein, 724 aa, Artificial Sequence
  Fusion protein of N-(L-methioninase from Pseudomonas putida)-Gly-Ser-Glye-Ser-Gly-Ser-(annexin V)-C
GPRDSHNNTGFSTRAIHHGYDPLSHGGALVPPVYQTATYAFPTVEYGAACFAGEEAGHFYSRISNPTLALLEQRMASLEGGEAGLALASGMGAITSTLWTLLRPGDELIVGRTLYGCTFAFLHHGIGEFGVKIHHVDLNDAKALKAAINSKTRMIYFETPANPNMQLVDIAAVVEAVRGSDVLVVVDNTYCTPYLQRPLELGADLVVHSATKYLSGHGDITAGLVVGRKALVDRIRLEGLKDMTGAALSPHDAALLMRGIKTLALRMDRHCANALEVAQFLAGQPQVELIHYPGLPSFAQYELAQRQMRLPGGMIAFELKGGIEAGRGFMNALQLFARAVSLGDAESLAQHPASMTHSSYTPQERAHHGISEGLVRLSVGLEDVEDLLADIELALEACAGSGSGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 24 - DNA, 2172 bp, Artificial Sequence
  DNA sequence of SEQ ID NO23
ggaccccgcgactcccataacaacaccggtttttccacacgggccattcaccacggctacgacccgctttcccacggtggtgccttggtgccaccggtgtaccagaccgcgacctatgccttcccgactgtcgaatacggcgctgcgtgcttcgccggggaggaggcggggcacttctacagccgcatctccaaccccaccctggccttgctcgagcaacgcatggcctcgttggagggtggtgaggcgggattggcgctggcgtcggggatgggagccattacttcgaccctctggaccctgctgcggcctggtgatgagctgatcgtggggcgcaccttgtatggctgcacctttgcgttcctgcaccatggcattggcgagttcggggtcaagatccaccatgtcgaccttaacgatgccaaggccctgaaagcggcgatcaacagcaaaacgcggatgatctacttcgaaacaccggccaaccccaacatgcaactggtggatatagcggcggtcgtcgaggcagtgcgggggagtgatgtgcttgtggtggtcgacaacacctactgcacgccctacctgcagcggccactggaactgggggcagacctggtggtgcattcggcaaccaagtacctcagtggccatggcgacatcactgcgggcctggtggtggggcgcaaggctttggtcgaccgcattcggctggaagggctgaaagacatgaccggggcagccttgtcaccgcatgacgctgcgttgttgatgcgcggcatcaagaccctggcgctgcgcatggaccggcattgcgccaacgccctggaggtcgcgcagttcctggccgggcagccccaggtggagctgatccactacccgggcttgccgtcgtttgcccagtacgaactggcacagcggcagatgcgtttgccgggcgggatgattgcctttgagctcaagggcggtatcgaggccgggcgcggcttcatgaatgccctgcagctttttgcccgtgcggtgagcctgggggatgccgagtcgctggcacagcacccggcgagcatgacgcactccagttacacgccacaagagcgggcgcatcacgggatatcagaggggctggtgaggttgtcagtggggctggaggatgtggaggacctgctggcagatatcgagttggcgttggaggcgtgtgcaggcagcggttctggatccgcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgac

SEQ ID NO: 25 - protein, 319 aa, Homo sapiens
AQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 26 - DNA, 525 bp, Homo sapiens
gcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgct

SEQ ID NO: 27 - protein, 4 aa, Artificial Sequence
  motif
HWGF

SEQ ID NO: 28 - protein, 10 aa, Artificial Sequence
  synthetic peptide
CTTHWGFTLC